SEQUENCE LISTING (105 sequences)

SEQ ID NO: 1 - DNA, 18 bp, Artificial Sequence
  Synthetic
cggtggacaaattgtcac

SEQ ID NO: 2 - DNA, 22 bp, Artificial Sequence
  Synthetic
cttctctggatttaacacactt

SEQ ID NO: 3 - DNA, 28 bp, Artificial Sequence
  Synthetic
ttacaagcttaaagaatgtctgaacact

SEQ ID NO: 4 - DNA, 27 bp, Artificial Sequence
  Synthetic
ttgaatttaggtgaaacatttgtcacg

SEQ ID NO: 5 - DNA, 47 bp, Artificial Sequence
  Synthetic
tcagcacacaaagccaaaaatttatctgtgcaaaggaaattaaggag

SEQ ID NO: 6 - DNA, 45 bp, Artificial Sequence
  Synthetic
tattggtggagctaaacttaaagccctgtacaatccctttgagtg

SEQ ID NO: 7 - DNA, 51 bp, Artificial Sequence
  Synthetic
tcagcacacaaagccaaaaatttatttttctgtgcaaaggaaattaaggag

SEQ ID NO: 8 - DNA, 49 bp, Artificial Sequence
  Synthetic
tattggtggagctaaacttaaagccttttctgtacaatccctttgagtg

SEQ ID NO: 9 - DNA, 18 bp, Artificial Sequence
  Synthetic
tgcttcagtcagctgatg

SEQ ID NO: 10 - DNA, 22 bp, Artificial Sequence
  Synthetic
ttaaattgtcatcttcgtcctt

SEQ ID NO: 11 - DNA, 41 bp, Artificial Sequence
  Synthetic
tcagtactagtgcctgtgcccacaatcgtttttaaacgggt

SEQ ID NO: 12 - DNA, 43 bp, Artificial Sequence
  Synthetic
tcgtatacagggcttttgacatctatcttggaagcgacaacaa

SEQ ID NO: 13 - DNA, 17 bp, Artificial Sequence
  Synthetic
ctgcacttacaccgcaa

SEQ ID NO: 14 - DNA, 22 bp, Artificial Sequence
  Synthetic
gtagctggttttgctaaattcc

SEQ ID NO: 15 - DNA, 17 bp, Artificial Sequence
  Synthetic
ttgatgagctggagcca

SEQ ID NO: 16 - DNA, 18 bp, Artificial Sequence
  Synthetic
caccctcaatgcagagtc

SEQ ID NO: 17 - DNA, 41 bp, Artificial Sequence
  Synthetic
gtgtgaccctgaagactcggttttagccactgactcggatc

SEQ ID NO: 18 - DNA, 45 bp, Artificial Sequence
  Synthetic
cctccgtgatatggctcttcgtttttttcttacatggctctggtc

SEQ ID NO: 19 - DNA, 20 bp, Artificial Sequence
  Synthetic
atgtggatggctgagttgtt

SEQ ID NO: 20 - DNA, 20 bp, Artificial Sequence
  Synthetic
catgctgagtactggacctc

SEQ ID NO: 21 - DNA, 13 bp, Artificial Sequence
  Synthetic
cagccatccacat

SEQ ID NO: 22 - DNA, 35 bp, Artificial Sequence
  Synthetic
tgtcggtctctggctccacaggtggctgccaatac

SEQ ID NO: 23 - DNA, 36 bp, Artificial Sequence
  Synthetic
cacgggagccactgactcccctgaagactcggatgt

SEQ ID NO: 24 - DNA, 15 bp, Artificial Sequence
  Synthetic
gcagcttcgggtcct

SEQ ID NO: 25 - DNA, 20 bp, Artificial Sequence
  Synthetic
ggataagtggaggagtgtct

SEQ ID NO: 26 - DNA, 18 bp, Artificial Sequence
  Synthetic
tcaacaagctccacggtg

SEQ ID NO: 27 - DNA, 17 bp, Artificial Sequence
  Synthetic
tccgcaacaactcagcc

SEQ ID NO: 28 - DNA, 51 bp, Artificial Sequence; identical to SEQ ID NO: 7
  Synthetic

SEQ ID NO: 29 - DNA, 49 bp, Artificial Sequence; identical to SEQ ID NO: 8
  Synthetic

SEQ ID NO: 30 - DNA, 18 bp, Artificial Sequence
  Synthetic
cggtggacaaattgtcac

SEQ ID NO: 31 - DNA, 22 bp, Artificial Sequence
  Synthetic
cttctctggatttaacacactt

SEQ ID NO: 32 - DNA, 28 bp, Artificial Sequence
  Synthetic
ttacaagcttaaagaatgtctgaacact

SEQ ID NO: 33 - DNA, 27 bp, Artificial Sequence
  Synthetic
ttgaatttaggtgaaacatttgtcacg

SEQ ID NO: 34 - DNA, 29 bp, Artificial Sequence
  Synthetic
ggtcgtagagcccattgcgcgatgagtgg

SEQ ID NO: 35 - DNA, 29 bp, Artificial Sequence
  Synthetic
ggtcgtagagcccattgcgcgatgagtgg

SEQ ID NO: 36 - DNA, 30 bp, Artificial Sequence
  Synthetic
cactgaccgaactgagctcctgaggcatgg

SEQ ID NO: 37 - DNA, 30 bp, Artificial Sequence; identical to SEQ ID NO: 36
  Synthetic

SEQ ID NO: 38 - DNA, 29 bp, Artificial Sequence
  Synthetic
ccactcatcgcgcaatgggctctacgacc

SEQ ID NO: 39 - DNA, 29 bp, Artificial Sequence
  Synthetic
ccactcatcgcgcaatgggctctacgacc

SEQ ID NO: 40 - DNA, 30 bp, Artificial Sequence
  Synthetic
ccatgcctcaggagctcagttcggtcagtg

SEQ ID NO: 41 - DNA, 30 bp, Artificial Sequence; identical to SEQ ID NO: 40
  Synthetic

SEQ ID NO: 42 - DNA, 23 bp, Artificial Sequence
  Synthetic
attgcgggagatgagacccgcaa

SEQ ID NO: 43 - DNA, 20 bp, Artificial Sequence
  Synthetic
caccggccaagacgcgccgg

SEQ ID NO: 44 - DNA, 35 bp, Artificial Sequence; identical to SEQ ID NO: 22
  Synthetic

SEQ ID NO: 45 - DNA, 36 bp, Artificial Sequence; identical to SEQ ID NO: 23
  Synthetic

SEQ ID NO: 46 - DNA, 15 bp, Artificial Sequence
  Synthetic
gcagcttcgggtcct

SEQ ID NO: 47 - DNA, 20 bp, Artificial Sequence
  Synthetic
ggataagtggaggagtgtct

SEQ ID NO: 48 - DNA, 18 bp, Artificial Sequence
  Synthetic
tcaacaagctccacggtg

SEQ ID NO: 49 - DNA, 17 bp, Artificial Sequence
  Synthetic
tccgcaacaactcagcc

SEQ ID NO: 50 - DNA, 18 bp, Artificial Sequence
  Synthetic
tcaacaagctccacggtg

SEQ ID NO: 51 - DNA, 17 bp, Artificial Sequence
  Synthetic
tccgcaacaactcagcc

SEQ ID NO: 52 - DNA, 51 bp, Artificial Sequence; identical to SEQ ID NO: 7
  Synthetic

SEQ ID NO: 53 - DNA, 49 bp, Artificial Sequence; identical to SEQ ID NO: 8
  Synthetic

SEQ ID NO: 54 - DNA, 18 bp, Artificial Sequence
  Synthetic
cggtggacaaattgtcac

SEQ ID NO: 55 - DNA, 22 bp, Artificial Sequence
  Synthetic
cttctctggatttaacacactt

SEQ ID NO: 56 - DNA, 28 bp, Artificial Sequence
  Synthetic
ttacaagcttaaagaatgtctgaacact

SEQ ID NO: 57 - DNA, 27 bp, Artificial Sequence
  Synthetic
ttgaatttaggtgaaacatttgtcacg

SEQ ID NO: 58 - DNA, 28 bp, Artificial Sequence
  Synthetic
ttacaagcttaaagaatgtctgaacact

SEQ ID NO: 59 - DNA, 27 bp, Artificial Sequence
  Synthetic
ttgaatttaggtgaaacatttgtcacg

SEQ ID NO: 60 - DNA, 29 bp, Artificial Sequence
  Synthetic
ccactcatcgcgcaatgggctctacgacc

SEQ ID NO: 61 - DNA, 48 bp, Artificial Sequence
  Synthetic
attgcgggagatgagacccgcaattgttggtcgtagagcccagaacga

SEQ ID NO: 62 - DNA, 29 bp, Artificial Sequence
  Synthetic
ccactcatctcgttctgggctctacgacc

SEQ ID NO: 63 - DNA, 48 bp, Artificial Sequence; identical to SEQ ID NO: 61
  Synthetic

SEQ ID NO: 64 - DNA, 30 bp, Artificial Sequence; identical to SEQ ID NO: 40
  Synthetic

SEQ ID NO: 65 - DNA, 45 bp, Artificial Sequence
  Synthetic
caccggccaagacgcgccggtgtgttcactgaccgaactggagca

SEQ ID NO: 66 - DNA, 56 bp, Artificial Sequence
  Synthetic
ggtcgtagagcccattgcgcgatgagtggttgaatttaggtgaaacatttgtcacg

SEQ ID NO: 67 - DNA, 56 bp, Artificial Sequence
  Synthetic
ggtcgtagagcccagaacgagatgagtggttgaatttaggtgaaacatttgtcacg

SEQ ID NO: 68 - DNA, 57 bp, Artificial Sequence
  Synthetic
cactgaccgaactgagctcctgaggcatggttgaatttaggtgaaacatttgtcacg

SEQ ID NO: 69 - DNA, 57 bp, Artificial Sequence
  Synthetic
ggtcgtagagcccattgcgcgatgagtggttacaagcttaaagaatgtctgaacact

SEQ ID NO: 70 - DNA, 57 bp, Artificial Sequence
  Synthetic
ggtcgtagagcccagaacgagatgagtggttacaagcttaaagaatgtctgaacact

SEQ ID NO: 71 - DNA, 58 bp, Artificial Sequence
  Synthetic
cactgaccgaactgagctcctgaggcatggttacaagcttaaagaatgtctgaacact

SEQ ID NO: 72 - DNA, 46 bp, Artificial Sequence
  Synthetic
ggtcgtagagcccattgcgcgatgagtggtccgcaacaactcagcc

SEQ ID NO: 73 - DNA, 46 bp, Artificial Sequence
  Synthetic
ggtcgtagagcccagaacgagatgagtggtccgcaacaactcagcc

SEQ ID NO: 74 - DNA, 47 bp, Artificial Sequence
  Synthetic
cactgaccgaactgagctcctgaggcatggtccgcaacaactcagcc

SEQ ID NO: 75 - DNA, 47 bp, Artificial Sequence
  Synthetic
ggtcgtagagcccattgcgcgatgagtggtcaacaagctccacggtg

SEQ ID NO: 76 - DNA, 47 bp, Artificial Sequence
  Synthetic
ggtcgtagagcccagaacgagatgagtggtcaacaagctccacggtg

SEQ ID NO: 77 - DNA, 48 bp, Artificial Sequence
  Synthetic
cactgaccgaactgagctcctgaggcatggtcaacaagctccacggtg

SEQ ID NO: 78 - DNA, 29 bp, Artificial Sequence
  Synthetic
ccactcatcgcgcaatgggctctacgacc

SEQ ID NO: 79 - DNA, 29 bp, Artificial Sequence
  Synthetic
ccactcatcgcgcaatgggctctacgacc

SEQ ID NO: 80 - DNA, 29 bp, Artificial Sequence
  Synthetic
ccactcatcgcgcaatgggctctacgacc

SEQ ID NO: 81 - DNA, 29 bp, Artificial Sequence
  Synthetic
ccactcatcgcgcaatgggctctacgacc

SEQ ID NO: 82 - DNA, 29 bp, Artificial Sequence
  Synthetic
ccactcatcgcgcaatgggctctacgacc

SEQ ID NO: 83 - DNA, 29 bp, Artificial Sequence
  Synthetic
ccactcatcgcgcaatgggctctacgacc

SEQ ID NO: 84 - DNA, 29 bp, Artificial Sequence
  Synthetic
ccactcatcgcgcaatgggctctacgacc

SEQ ID NO: 85 - DNA, 29 bp, Artificial Sequence
  Synthetic
ccactcatctcgttctgggctctacgacc

SEQ ID NO: 86 - DNA, 29 bp, Artificial Sequence
  Synthetic
ccactcatctcgttctgggctctacgacc

SEQ ID NO: 87 - DNA, 29 bp, Artificial Sequence
  Synthetic
ccactcatctcgttctgggctctacgacc

SEQ ID NO: 88 - DNA, 29 bp, Artificial Sequence
  Synthetic
ccactcatctcgttctgggctctacgacc

SEQ ID NO: 89 - DNA, 29 bp, Artificial Sequence
  Synthetic
ccactcatctcgttctgggctctacgacc

SEQ ID NO: 90 - DNA, 29 bp, Artificial Sequence
  Synthetic
ccactcatctcgttctgggctctacgacc

SEQ ID NO: 91 - DNA, 29 bp, Artificial Sequence
  Synthetic
catgcctcaggagctcagttcggtcagtg

SEQ ID NO: 92 - DNA, 29 bp, Artificial Sequence
  Synthetic
catgcctcaggagctcagttcggtcagtg

SEQ ID NO: 93 - DNA, 29 bp, Artificial Sequence
  Synthetic
catgcctcaggagctcagttcggtcagtg

SEQ ID NO: 94 - DNA, 30 bp, Artificial Sequence; identical to SEQ ID NO: 40
  Synthetic

SEQ ID NO: 95 - DNA, 30 bp, Artificial Sequence; identical to SEQ ID NO: 40
  Synthetic

SEQ ID NO: 96 - DNA, 30 bp, Artificial Sequence; identical to SEQ ID NO: 40
  Synthetic

SEQ ID NO: 97 - DNA, 30 bp, Artificial Sequence; identical to SEQ ID NO: 40
  Synthetic

SEQ ID NO: 98 - DNA, 48 bp, Artificial Sequence; identical to SEQ ID NO: 61
  Synthetic

SEQ ID NO: 99 - DNA, 48 bp, Artificial Sequence; identical to SEQ ID NO: 61
  Synthetic

SEQ ID NO: 100 - DNA, 48 bp, Artificial Sequence; identical to SEQ ID NO: 61
  Synthetic

SEQ ID NO: 101 - DNA, 45 bp, Artificial Sequence; identical to SEQ ID NO: 65
  Synthetic

SEQ ID NO: 102 - DNA, 45 bp, Artificial Sequence; identical to SEQ ID NO: 65
  Synthetic

SEQ ID NO: 103 - DNA, 45 bp, Artificial Sequence; identical to SEQ ID NO: 65
  Synthetic

SEQ ID NO: 104 - DNA, 24 bp, Artificial Sequence
  Synthetic
tgttggtcgtagagcccagaacga

SEQ ID NO: 105 - DNA, 24 bp, Artificial Sequence
  Synthetic
gtgttcactgaccgaactggagca

(19) United States
(12) Patent Application Publication
(10) Pub. No.: US 2022/0010386 A1
(43) Pub. Date: Jan. 13, 2022

(54) RAPID DIAGNOSTIC TEST

(71) Applicant: Detect, Inc., Guilford, CT (US)

(72) Inventors: Henry Kemble, Paris (FR); Spencer Glantz, West Hartford, CT (US); Jeffrey Sabina, Hamden, CT (US); Sebastian Jaramillo-Riveri, New Haven, CT (US)

(73) Assignee: Detect, Inc., Guilford, CT (US)

(21) Appl. No.: 17/371,640

(22) Filed: Jul. 9, 2021

Related U.S. Application Data
(60) Provisional application No. 63/089,801, filed on Oct. 9, 2020.
(60) Provisional application No. 63/086,196, filed on Oct. 1, 2020.
(60) Provisional application No. 63/050,659, filed on Jul. 10, 2020.

(51) Int. Cl. C12Q 1/6888; C12Q 1/6818; C12Q 1/6844

(52) U.S. Cl. CPC C12Q 1/6888; C12Q 2600/158; C12Q 1/6846; C12Q 1/6818

(57) ABSTRACT

Aspects of the disclosure relate to compositions and methods for amplifying and/or detecting one or more target nucleic acid sequences (e.g., a nucleic acid sequence of one or more pathogens) in a biological sample obtained from a subject. In some embodiments, the pathogens are viral, bacterial, fungal, parasitic, or protozoan pathogens, such as SARS-CoV-2 or an influenza virus. In some embodiments, the methods comprise isothermal amplification of a target nucleic acid and subsequent detection of the amplification products.

### RELATED APPLICATIONS

This application claims the benefit under 35 U.S.C. 119(e) of the filing date of U.S. provisional application serial numbers U.S. 63/050,659, filed Jul. 10, 2020, U.S. 63/089,801, filed Oct. 9, 2020, and U.S. 63/086,196, filed Oct. 1, 2020, the entire contents of each of which are incorporated herein by reference.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 9, 2021, is named H096670039US03-SEQ-KZM and is 19,465 bytes in size.

### FIELD

The disclosure generally relates to diagnostic devices, systems, and methods for detecting the presence of a target nucleic acid sequence.

### BACKGROUND

The ability to rapidly diagnose diseases-particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Aspects of the disclosure relate to compositions and methods for amplifying and/or detecting target analytes (e.g., nucleic acids) in a sample. The disclosure is based, in part, on methods and compositions that produce amplification products that result in high contrast, visible, colored bands when bound to certain immunoassay devices (e.g., lateral flow immunoassays). In some embodiments, compositions and methods described herein allow for direct application of the amplicons to immunoassay devices without any intervening fluid transfer or dilution steps.

In some aspects, the disclosure describes several distinct methods for the direct application of reacted nucleic acid amplification (NAA) mixes to lateral flow strips without any intervening dilution or sample preparation steps. In some embodiments, the disclosed methods incorporate secondary nucleic acid reactions alongside the primary NAA reaction responsible for amplifying the diagnostic target sequence. These secondary nucleic acid reactions result in the formation of controllable amounts of secondary products of desired size, structure and chemical labelling, position, and/or stoichiometry, which function as ideal lateral flow assay (LFA) analytes according to these properties. In some embodiments, methods described herein enable direct readout of the sensitive and specific detection of a target nucleic acid by convenient one-step sandwich assay devices like LFA strips. In some embodiments, methods described herein also permit sensitive multiplexed LFA detection.

The disclosure also relates, in some aspects, to compositions, and kits, such as test kits, to implement the methods described herein. Such methods, compositions, and kits have several advantages over currently used diagnostic assays, for example mitigation of the LFA ‘hook-effect’ without compromising assay sensitivity or speed; high-specificity detection of isothermal amplification reactions; improved variant detection; and sequence-independent ‘universal’ applicability across diagnostic areas (e.g., infectious diseases, etc.).

Accordingly, in some aspects, the disclosure provides an oligonucleotide strand displacement probe comprising a first single stranded polynucleotide having a 5′ portion and a 3′ portion, wherein the 5′ portion comprises a first hapten and the 3′ portion comprises an extension blocking group; and a second single stranded polynucleotide comprising a 5′ portion and a 3′ portion, wherein the 5′ portion comprises a biotin, wherein the second single stranded polynucleotide is hybridized to the first single stranded polynucleotide to form a duplex region and a single stranded toehold region comprising the 3′ portion of the first single stranded polynucleotide.

In some embodiments, a first single stranded polynucleotide ranges from about 10 to about 50 nucleotides in length. In some embodiments, a stranded polynucleotide ranges from about 10 to about 50 nucleotides in length.

In some embodiments, a duplex region ranges from about 5 nucleotides to about 40 nucleotides in length. In some embodiments, a duplex region comprises a blunt end.

In some embodiments, a single stranded toehold region ranges from about 5 to about 45 nucleotides in length. In some embodiments, a single stranded toehold region comprises a region of complementarity with a target gene.

In some embodiments, a target gene is derived from a pathogen. In some embodiments, a pathogen is a viral, bacterial, fungal, parasitic, or protozoan pathogen. In some embodiments, a viral pathogen is SARS-CoV-2.

In some embodiments, a first hapten comprises FITC.

In some embodiments, an extension blocking group comprises a dehydroxylated 3′ nucleotide, 3′ dideoxycytosine (ddC), 3′ inverted deoxythymidine (dT), 3′ propyl (C3) spacer, 3′ amino modification, or 3′ phosphoryl modification.

In some aspects, the disclosure provides an amplification mixture comprising an oligonucleotide strand displacement probe as described herein, and one or more of the following: one or more buffering agents (e.g., Tris-HCl, tricine, bicine, (NH₄)2SO₄, etc.), one or more salts (e.g., KCl, MgSO₄, etc.) and one or more detergents (e.g., Tween20, Triton-X-100, etc.); an outer primer pair comprising a region of complementarity with a target nucleic acid; an internal primer pair comprising a region of complementarity with the target nucleic acid; a first loop primer pair comprising a first loop primer linked to a biotin and a second loop primer linked to a first hapten, wherein the first and second loop primers each comprise a region of complementarity with the target nucleic acid; a deoxynucleoside triphosphate (dNTP) mixture; a polymerase; and a reverse transcriptase.

In some aspects, the disclosure provides an amplification mixture comprising: one or more buffering agents (e.g., Tris-HCl, tricine, bicine, (NH₄)2SO₄, etc.), one or more salts (e.g., KCl, MgSO₄, etc.) and one or more detergents (e.g., Tween20, Triton-X-100, etc.); an outer primer pair comprising a region of complementarity with a target nucleic acid; an internal primer pair comprising a region of complementarity with the target nucleic acid; a first loop primer pair comprising a first loop primer linked to a biotin and a second loop primer linked to a first hapten, wherein the first and second loop primers each comprise a region of complementarity with the target nucleic acid; an oligonucleotide strand displacement probe comprising a first single stranded polynucleotide having a 5′ portion and a 3′ portion, wherein the 3′ portion comprises an extension blocking group; and a second single stranded polynucleotide comprising a 5′ portion and a 3′ portion, wherein the 5′ portion comprises a biotin and the 3′ portion comprises an extension blocking group, and wherein the second single stranded polynucleotide is hybridized to the first single stranded polynucleotide to form a duplex region and a single stranded toehold region comprising the 3′ portion of the first single stranded polynucleotide; a single stranded reporter oligonucleotide that is complementary to the second single stranded polynucleotide of (e)(ii); a deoxynucleoside triphosphate (dNTP) mixture; a polymerase; and a reverse transcriptase.

In some embodiments, a 5′ portion of a first single stranded polynucleotide comprises a digoxigenin (DIG).

In some embodiments, a single stranded reporter oligonucleotide comprises a FITC on its 5′ portion or 3′ portion. In some embodiments, a single stranded reporter oligonucleotide comprises an extension blocking group on its 5′ portion or 3′ portion.

In some aspects, the disclosure provides a method for detecting target nucleic acids in a biological sample, the method comprising contacting an amplification mixture as described herein with a biological sample; performing an isothermal amplification reaction on the mixture to produce an amplification mixture; contacting the amplification mixture to a lateral flow assay (LFA) device under conditions under which the oligonucleotide strand displacement probe in the amplification mixture produces a first detectable signal when a target nucleotide is absent from the biological sample and/or a second detectable signal when a control nucleic acid is present in the biological sample; and identifying the presence of the target nucleic acid in the biological sample based upon detecting the absence of a first detectable signal and the presence of a second detectable signal, or identifying the absence of the target nucleic acid in the biological sample based on detecting the presence of the first detectable signal and the presence of the second detectable signal.

In some aspects, the disclosure provides a method for detecting target nucleic acids in a biological sample, the method comprising contacting an amplification mixture as described herein with a biological sample; performing an isothermal amplification reaction on the mixture to produce an amplification mixture; contacting the amplification mixture to a lateral flow assay (LFA) device under conditions under which the single stranded reporter oligonucleotide in the amplification mixture produces a first detectable signal when a target nucleotide is present from the biological sample or a second detectable signal when the target nucleic acid is absent from the biological sample; and identifying the presence of the target nucleic acid in the biological sample based upon detecting the presence of a first detectable signal and the absence of a second detectable signal, or identifying the absence of the target nucleic acid in the biological sample based on detecting the absence of the first detectable signal and the presence of the second detectable signal.

In some embodiments, a biological sample comprises blood, saliva, mucous, urine, feces, cerebrospinal fluid (CSF), or tissue.

In some embodiments, a target nucleic acid is derived from a pathogen. In some embodiments, a pathogen is a viral, bacterial, fungal, parasitic, or protozoan pathogen. In some embodiments, a viral pathogen is SARS-CoV-2.

In some embodiments, methods described herein further comprise a step of detecting the presence of one or more control nucleic acids. In some embodiments, one or more control nucleic acids are human nucleic acids.

In some aspects, the disclosure provides a mediator displacement probe comprising a hemi-duplex nucleic acid comprising a first single stranded polynucleotide having a 5′ end and a 3′ end, comprising a target sequence-specific portion contiguously linked to a mediator complement (MedC) portion; and a second single stranded polynucleotide having a 5′ end and a 3′ end, comprising a mediator (Med) polynucleotide sequence, a label attached to the 5′ end of the Med polynucleotide sequence, and an extension blocking agent attached to the 3′ end of the Med polynucleotide sequence, wherein the MedC portion of the first single stranded polynucleotide and the Med polynucleotide sequence of the second single stranded polynucleotide are annealed together to form a hemi-duplex molecule, wherein the target sequence-specific portion of the first single stranded polynucleotide forms the single stranded portion of the hemi-duplex molecule.

In some embodiments, a first single stranded polynucleotide ranges from about 10 to about 50 nucleotides in length. In some embodiments, a second stranded polynucleotide ranges from about 10 to about 50 nucleotides in length.

In some embodiments, a duplex region of a hemi-duplex molecule ranges from about 5 nucleotides to about 40 nucleotides in length. In some embodiments, a duplex region of a hemi-duplex molecule comprises a blunt end.

In some embodiments a single stranded portion ranges from about 5 to about 45 nucleotides in length.

In some embodiments, a target sequence-specific portion comprises a region of complementarity to a human gene. In some embodiments, a human gene encodes RNAse P.

In some embodiments, a target sequence specific portion comprises a region of complementarity to a target gene derived from a pathogen. In some embodiments, a pathogen is a viral, bacterial, fungal, parasitic, or protozoan pathogen. In some embodiments, a pathogen is SARS-CoV-2.

In some embodiments, a target sequence-specific portion is positioned 5′ relative to the MedC portion. In some embodiments, a MedC portion comprises the sequence set forth in any one of SEQ ID NOs: 34-37.

In some embodiments, a Med polynucleotide sequence comprises the sequence set forth in any one of SEQ ID NOs: 38-41.

In some embodiments, a label is selected from FAM, FITC, digoxigenin (DIG), and biotin.

In some embodiments, an extension blocking agent is selected from biotin, a dehydroxylated 3′ nucleotide, 3′ dideoxycytosine (ddC), 3′ inverted deoxythymidine (dT), 3′ propyl (C3) spacer, 3′ amino modification, and a 3′ phosphoryl modification.

In some embodiments, the disclosure provides a mediator displacement probe set, the probe set comprising a mediator displacement probe as described herein; and a Universal Reporter hairpin polynucleotide having a 5′ end and a 3′ end, comprising a label attached to the 5′ end, and single stranded polynucleotide sequence that is complementary to the mediator (Med) polynucleotide sequence of the mediator displacement probe of (i) positioned at the 3′ end.

In some embodiments, a Universal Reporter hairpin polynucleotide ranges from about 30 to about 100 nucleotides in length.

In some embodiments, a label attached to the 5′ end is selected from FAM, FITC, digoxigenin (DIG), and biotin.

In some embodiments, a single stranded polynucleotide sequence complementary to the Med polynucleotide sequence is identical to (e.g., shares 100% sequence identity with) a MedC portion of the mediator displacement probe.

In some embodiments, a single stranded polynucleotide sequence is complementary to a Med polynucleotide sequence comprises the sequence set forth in any one of SEQ ID NOs: 34-37.

In some embodiments, a mediator displacement probe set further comprises one or more LAMP primers. In some embodiments, one or more LAMP primers comprise an outer primer pair comprising a region of complementarity with a target nucleic acid; an internal primer pair comprising a region of complementarity with the target nucleic acid; and a first loop primer pair comprising a first loop primer and a second loop primer, wherein the first and second loop primers each comprise a region of complementarity with the target nucleic acid. In some embodiments, a target sequence-specific portion of a mediator displacement probe is identical to a region of complementarity of the loop primers.

In some aspects, the disclosure provides an amplification mixture comprising a mediator displacement probe set as described herein; one or more buffering agents (e.g., Tris-HCl, tricine, bicine, (NH4)2SO4, etc.), one or more salts (e.g., KCl, MgSO4, etc.) and one or more detergents (e.g., Tween20, Triton-X-100, etc.); a deoxynucleoside triphosphate (dNTP) mixture; a polymerase; and a reverse transcriptase.

In some embodiments, an amplification mixture further comprises a target nucleic acid. In some embodiments, a target nucleic acid is present in (or isolated from) a biological sample obtained from a subject. In some embodiments, a subject is a human. In some embodiments, a target nucleic acid is derived from a pathogen. In some embodiments, a pathogen is a viral, bacterial, fungal, parasitic, or protozoan pathogen. In some embodiments, a pathogen is SARS-CoV-2.

In some aspects, the disclosure provides a method for producing dual-labeled lateral flow assay (LFA) detection products, the method comprising performing an isothermal amplification reaction to amplify a target nucleic acid in the presence of a mediator displacement probe set as described herein; and a Universal Reporter hairpin polynucleotide having a 5′ end and a 3′ end, comprising a label attached to the 5′ end, and single stranded polynucleotide sequence that is complementary to the mediator (Med) polynucleotide sequence of the mediator displacement probe of (i) positioned at the 3′ end; and producing dual-labeled LFA detection products by integrating the mediator displacement probe into an amplicon of the isothermal amplification reaction, thereby releasing the Med portion of the mediator displacement probe, which subsequently anneals to the Universal Reporter hairpin polynucleotide to produce a dual-labeled LFA detection product.

In some embodiments, an isothermal amplification reaction is LAMP.

In some embodiments, a mediator displacement probe comprises a target sequence-specific portion that is identical to a Loop primer of the isothermal amplification reaction. In some embodiments, a Loop primer is a forward Loop primer or a reverse Loop primer.

In some embodiments, the concentration ratio of a mediator displacement probe to a Universal Reporter hairpin polynucleotide ranges from about 3:1 to about 1:3. In some embodiments, a concentration of a mediator displacement probe in an amplification reaction ranges from about 0.4 nM to about 500 nM. In some embodiments, the concentration of a Universal Reporter hairpin polynucleotide in an amplification reaction ranges from about 0.4 nM to about 500 nM.

In some embodiments, production of dual-labeled LFA detection products is indicative of amplification of a target nucleic acid by the isothermal amplification reaction.

In some embodiments, methods described herein further comprise amplifying one or more additional target nucleic acids, and wherein the amplification is performed in the presence of one or more additional mediator displacement probe sets corresponding to said additional target nucleic acids.

In some embodiments, one or more additional target nucleic acids comprises a control nucleic acid. In some embodiments, the presence of the one or more additional mediator displacement probe sets results in production of one or more additional dual-labeled LFA detection products.

In some embodiments, the disclosure provides a method for indirectly detecting isothermal amplification of a target nucleic acid, the method comprising producing one or more dual-labeled LFA detection products according to a method as described herein; contacting a lateral flow assay (LFA) device with the one or more dual-labeled LFA amplification products to produce one or more detectable signals; and identifying the presence of the target nucleic acid in the isothermal amplification based upon detecting the presence of the detectable signal produced by the one or more dual-labeled LFA detection products.

In some embodiments, a target nucleic acid is derived from a pathogen. In some embodiments, a pathogen is a viral, bacterial, fungal, parasitic, or protozoan pathogen. In some embodiments, a pathogen is SARS-CoV-2.

Further aspects of the disclosure relate to methods for increasing LFA band intensity after isothermal amplification reactions by altering amplification primer concentration and/or ratio. In some aspects, the disclosure provides a method for detecting target nucleic acids in a biological sample, the method comprising performing an isothermal amplification reaction on a biological sample comprising a target gene and a control gene, wherein the amplification reaction is performed in a buffer solution comprising one or more biotinylated oligonucleotides, a target nucleic acid-specific primer comprising a first hapten, and a control nucleic acid-specific primer comprising a second hapten different from the first hapten, wherein the buffer comprises a total concentration of biotinylated oligonucleotides that is less than about 200 nM, a total concentration of first hapten less than or equal to about 25 nM, and a total concentration of second hapten that is less than or equal to about 100 nM, thereby producing an amplification product mixture comprising labeled target amplification products and labeled control amplification products; contacting a lateral flow assay (LFA) device comprising a first test line that specifically binds to the first hapten and a second test line that specifically binds to the second hapten with an undiluted aliquot of the amplification product mixture under conditions under which the labeled target amplification products bind to the first test line to produce a first detectable signal or the labeled control amplification products bind to the second test line to produce a second detectable signal; and identifying the presence of the target nucleic acid in the biological sample based upon detecting the presence of the first detectable signal and the second detectable signal, or identifying the absence of the target nucleic acid in the biological sample based on detecting the absence of the first detectable signal and the presence of the second detectable signal. In some embodiments, the method is performed only on a target gene (e.g., a control gene is not amplified).

In some embodiments, a target nucleic acid comprises DNA or RNA. In some embodiments, a target nucleic acid is derived from a pathogen. In some embodiments, a pathogen is a viral, bacterial, fungal, parasitic, or protozoan pathogen. In some embodiments, a control nucleic acid is derived from a different species than a target nucleic acid. In some embodiments, a control nucleic acid is derived from a human.

In some embodiments, a biological sample comprises blood, saliva, mucous, urine, feces, cerebrospinal fluid (CSF), or tissue. In some embodiments, a biological sample is obtained from a subject. In some embodiments, a subject is a mammal. In some embodiments, a subject is a human.

In some embodiments, an isothermal amplification reaction comprises loop-mediated isothermal amplification (LAMP). In some embodiments, LAMP comprises reverse transcription LAMP (RT-LAMP).

In some embodiments, an isothermal amplification reaction (e.g., RT-LAMP) is performed using a polymerase selected from the group consisting of Bacillus stearothermophilus (Bst), Bacillus Smithii (Bsm), Geobacillus sp. M (GspM), Thermodesulfatator indicus (Tin), and Taq DNA polymerase.

In some embodiments, a reverse transcriptase used for an isothermal amplification reaction (e.g., RT-LAMP) is selected from the group consisting of HIV-1 reverse transcriptase, Moloney murine leukemia virus (M-MLV) reverse transcriptase, and avian myeloblastosis virus (AMV) reverse transcriptase.

In some embodiments, one or more biotinylated oligonucleotides comprise a target nucleic acid-specific biotinylated primer and/or a control nucleic acid-specific biotinylated primer. In some embodiments, a target nucleic acid-specific biotinylated primer and a control nucleic acid-specific biotinylated primer are present in non-equimolar concentrations in a buffer solution. In some embodiments, the ratio of concentration of a target nucleic acid-specific biotinylated primer to a control nucleic acid-specific biotinylated primer ranges from about 1:5 to about 5:1. In some embodiments, the ratio of concentration of a target nucleic acid-specific biotinylated primer to a control nucleic acid-specific biotinylated primer ranges from about 1:3 to about 3:1.

In some embodiments, a first hapten comprises a Fluorescein isothiocyanate (FITC) molecule. In some embodiments, the concentration of a first hapten in a buffer solution ranges from about 5 nM to about 25 nM.

In some embodiments, a second hapten comprises a digoxigenin (DIG) molecule. In some embodiments, the concentration of a second hapten in a buffer solution ranges from about 5 nM to about 100 nM.

In some embodiments, a buffer solution further comprises one or more sets (e.g., pairs) of outer primers. In some embodiments, at least one set (e.g. pair) of outer primers specifically binds to a target nucleic acid. In some embodiments, at least one set of outer primers specifically binds to a control nucleic acid.

In some embodiments, a buffer solution further comprises one or more sets (e.g., pairs) of internal primers. In some embodiments, at least one set (e.g., pair) of internal primers specifically binds to a target nucleic acid. In some embodiments, at least one set (e.g., pair) of internal primers specifically binds to a control nucleic acid.

In some embodiments, an amplification reaction proceeds for less than 45 minutes.

In some embodiments, identifying comprises visually confirming the presence of a first detectable signal and/or a second detectable signal.

In some embodiments, methods described herein further comprise the step of identifying a subject as being infected with a pathogen based upon the presence of a target nucleic acid.

In some embodiments, methods described herein further comprise the step of administering one or more therapeutic agents to a subject. In some embodiments, the one or more therapeutic agents are antiviral agents.

In some aspects, the disclosure provides a method for amplifying a target nucleic acid, the method comprising: contacting a target nucleic acid in biological sample obtained from a subject with an amplification buffer to produce an amplification mixture, wherein the amplification buffer comprises: one or more buffering agents (e.g., Tris-HCl, tricine, bicine, (NH4)2SO₄, etc.), one or more salts (e.g., KCl, MgSO₄, etc.) and one or more detergents (e.g., Tween20, Triton-X-100, etc.); an outer primer pair comprising a region of complementarity with the target nucleic acid; an internal primer pair comprising a region of complementarity with the target nucleic acid; a first loop primer pair comprising a first loop primer linked to a biotin and a second loop primer linked to a first hapten, wherein the first and second loop primers each comprise a region of complementarity with the target nucleic acid, and wherein the concentration of the first loop primer in the amplification mixture is less than or equal to 200 nM and the concentration of the second loop primer in the amplification mixture is less than or equal to 100 nM; a deoxynucleoside triphosphate (dNTP) mixture; a polymerase; and a reverse transcriptase; and performing an isothermal amplification reaction on the amplification mixture to produce a composition comprising an increased amount of target nucleic acid relative to the amount of target nucleic acid in the biological sample.

In some embodiments, a target nucleic acid is DNA or RNA. In some embodiments, a biological sample comprises blood, saliva, mucous, urine, feces, cerebrospinal fluid (CSF), or tissue. In some embodiments, a biological sample further comprises a control nucleic acid and an amplification buffer further comprises: a second outer primer pair comprising a region of complementarity with the control nucleic acid; a second internal primer pair comprising a region of complementarity with the control nucleic acid; and/or a second loop primer pair comprising a third loop primer linked to a biotin and a fourth loop primer linked to a second hapten, wherein the third and fourth loop primers each comprise a region of complementarity with the control nucleic acid.

In some embodiments, a first hapten comprises FITC. In some embodiments, a second hapten comprises digoxigenin (DIG).

In some embodiments, a first loop primer and a third loop primer are present in non-equimolar concentrations in an amplification buffer.

In some embodiments, the concentration of a second loop primer in an amplification buffer ranges from about 5 nM to about 25 nM. In some embodiments, the concentration of a fourth loop primer in an amplification buffer ranges from about 5 nM to about 100 nM.

In some embodiments, a dNTP mixture comprises deoxyadenosine 5′-triphosphate (dATP), deoxyguanine 5′-triphosphate (dGTP), deoxythymidine 5′-triphosphate (dTTP), deoxycytidine 5′-triphosphate (dCTP), and 2′-Deoxyuridine 5′-Triphosphate (dUTP).

In some embodiments, a polymerase is selected from the group consisting of Bacillus stearothermophilus (Bst), Bacillus Smithii (Bsm), Geobacillus sp. M (GspM), Thermodesulfatator indicus (Tin), and Taq DNA polymerase.

In some embodiments, a reverse transcriptase is selected from the group consisting of HIV-1 reverse transcriptase, Moloney murine leukemia virus (M-MLV) reverse transcriptase, and avian myeloblastosis virus (AMV) reverse transcriptase.

In some embodiments, an isothermal amplification reaction comprises loop-mediated isothermal amplification (LAMP). In some embodiments, LAMP comprises reverse transcription LAMP (RT-LAMP).

In some embodiments, a target nucleic acid is derived from a pathogen. In some embodiments, a pathogen is a viral, bacterial, fungal, parasitic, or protozoan pathogen. In some embodiments, a viral pathogen is SARS-CoV-2.

In some embodiments, a control nucleic acid is derived from a mammal. In some embodiments, a control nucleic acid is derived from a human.

In some aspects, the disclosure provides a method for preparing a population of nucleic acid amplification products useful for analyzing a target gene in a biological sample obtained from a subject, the method comprising performing an isothermal amplification reaction on a biological sample comprising a target gene and a control gene, wherein the amplification reaction is performed in a buffer solution comprising one or more biotinylated oligonucleotides, a target nucleic acid-specific primer comprising a first hapten, and a control nucleic acid-specific primer comprising a second hapten different from the first hapten, wherein the buffer comprises a total concentration of biotinylated oligonucleotides that is less than 200 nM, a total concentration of first hapten less than or equal to 25 nM, and a total concentration of second hapten that is less than or equal to 100 nM, thereby producing an amplification product mixture comprising labeled target amplification products and labeled control amplification products; and analyzing a target gene in the amplification product mixture.

In some embodiments, analyzing comprises contacting a lateral flow assay (LFA) device comprising a first test line that specifically binds to a first hapten and a second test line that specifically binds to a second hapten with an undiluted aliquot of an amplification product mixture under conditions under which labeled target amplification products bind to the first test line to produce a first detectable signal or labeled control amplification products bind to the second test line to produce a second detectable signal. In some embodiments, analyzing further comprises identifying the presence of a target nucleic acid in a biological sample based upon detecting the presence of a first detectable signal and a second detectable signal, or identifying the absence of a target nucleic acid in a biological sample based on detecting the absence of a first detectable signal and the presence of a second detectable signal.

In some embodiments, methods described herein further comprising the step of identifying a subject from which a biological sample was obtained as being infected with an organism encoding a target nucleic acid when the target nucleic acid is present in the amplification product mixture.

In some aspects, the disclosure provides an amplification mixture comprising one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.); an outer primer pair comprising a region of complementarity with the target nucleic acid; an internal primer pair comprising a region of complementarity with the target nucleic acid; a first loop primer pair comprising a first loop primer linked to a biotin and a second loop primer linked to a first hapten, wherein the first and second loop primers each comprise a region of complementarity with the target nucleic acid, and wherein the concentration of the first loop primer in the amplification mixture is less than or equal to 200 nM and the concentration of the second loop primer in the amplification mixture is less than or equal to 100 nM; a deoxynucleoside triphosphate (dNTP) mixture; a polymerase; and a reverse transcriptase.

In some embodiments, an amplification mixture further comprises a biological sample. In some embodiments, a biological sample comprises blood, saliva, mucous, urine, feces, cerebrospinal fluid (CSF), or tissue. In some embodiments, a biological sample comprises a target nucleic acid. In some embodiments, a target nucleic acid is derived from a pathogen. In some embodiments, a pathogen is a viral, bacterial, fungal, parasitic, or protozoan pathogen. In some embodiments, a viral pathogen is SARS-CoV-2.

In some embodiments, an amplification mixture further comprises a control nucleic acid. In some embodiments, a control nucleic acid is derived from a human.

In some embodiments, an amplification primer further comprises: a second outer primer pair comprising a region of complementarity with the control nucleic acid; a second internal primer pair comprising a region of complementarity with the control nucleic acid; and/or a second loop primer pair comprising a third loop primer linked to a biotin and a fourth loop primer linked to a second hapten, wherein the third and fourth loop primers each comprise a region of complementarity with the control nucleic acid.

In some embodiments, a dNTP mixture comprises deoxyadenosine 5′-triphosphate (dATP), deoxyguanine 5′-triphosphate (dGTP), deoxythymidine 5′-triphosphate (dTTP), deoxycytidine 5′-triphosphate (dCTP), and 2′-Deoxyuridine 5′-Triphosphate (dUTP).

In some embodiments, a polymerase is selected from the group consisting of Bacillus stearothermophilus (Bst), Bacillus Smithii (Bsm), Geobacillus sp. M (GspM), Thermodesulfatator indicus (Tin), and Taq DNA polymerase.

In some embodiments, a reverse transcriptase is selected from the group consisting of HIV-1 reverse transcriptase, Moloney murine leukemia virus (M-MLV) reverse transcriptase, and avian myeloblastosis virus (AMV) reverse transcriptase.

In some embodiments, the disclosure comprises a container comprising an amplification mixture as described herein.

In some aspects, the disclosure provides an oligonucleotide strand displacement probe comprising a first single stranded polynucleotide having a 5′ portion and a 3′ portion, wherein the 5′ portion comprises a first hapten and the 3′ portion comprises an extension blocking group; and a second single stranded polynucleotide comprising a 5′ portion and a 3′ portion, wherein the 5′ portion comprises a biotin, wherein the second single stranded polynucleotide is hybridized to the first single stranded polynucleotide to form a duplex region and a single stranded toehold region comprising the 3′ portion of the first single stranded polynucleotide.

In some embodiments, a first single stranded polynucleotide ranges from about 10 to about 50 nucleotides in length. In some embodiments, a second single stranded polynucleotide ranges from about 10 to about 50 nucleotides in length.

In some embodiments, a duplex region ranges from about 5 nucleotides to about 40 nucleotides in length. In some embodiments, a duplex region comprises a blunt end.

In some embodiments, a single stranded toehold region ranges from about 5 to about 45 nucleotides in length. In some embodiments, a single stranded toehold region comprises a region of complementarity with a target gene. In some embodiments, a target gene is derived from a pathogen. In some embodiments, a pathogen is a viral, bacterial, fungal, parasitic, or protozoan pathogen. In some embodiments, a viral pathogen is SARS-CoV-2.

In some embodiments, a first hapten comprises FITC. In some embodiments, an extension blocking group comprises a dehydroxylated 3′ nucleotide, 3′ dideoxycytosine (ddC), 3′ inverted deoxythymidine (dT), 3′ propyl (C3) spacer, 3′ amino modification, or 3′ phosphoryl modification.

In some aspects, the disclosure provides an amplification mixture comprising an oligonucleotide strand displacement probe as described herein, and one or more of the following: one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.); an outer primer pair comprising a region of complementarity with a target nucleic acid; an internal primer pair comprising a region of complementarity with the target nucleic acid; a first loop primer pair comprising a first loop primer linked to a biotin and a second loop primer linked to a first hapten, wherein the first and second loop primers each comprise a region of complementarity with the target nucleic acid; a deoxynucleoside triphosphate (dNTP) mixture; a polymerase; and a reverse transcriptase.

In some aspects, the disclosure provides an amplification mixture comprising: one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.); an outer primer pair comprising a region of complementarity with a target nucleic acid; an internal primer pair comprising a region of complementarity with the target nucleic acid; a first loop primer pair comprising a first loop primer linked to a biotin; and a second loop primer linked to a first hapten, wherein the first and second loop primers each comprise a region of complementarity with the target nucleic acid; an oligonucleotide strand displacement probe comprising a first single stranded polynucleotide having a 5′ portion and a 3′ portion, wherein the 3′ portion comprises an extension blocking group; and a second single stranded polynucleotide comprising a 5′ portion and a 3′ portion, wherein the 5′ portion comprises a biotin, and the 3′ portion comprises an extension blocking group, and wherein the second single stranded polynucleotide is hybridized to the first single stranded polynucleotide to form a duplex region and a single stranded toehold region comprising the 3′ portion of the first single stranded polynucleotide; a single stranded reporter oligonucleotide that is complementary to the second single stranded polynucleotide of (e)(ii); a deoxynucleoside triphosphate (dNTP) mixture; a polymerase; and a reverse transcriptase.

In some embodiments, a 5′ portion of the first single stranded polynucleotide of (e)(i) comprises a digoxigenin (DIG).

In some embodiments, a single stranded reporter oligonucleotide comprises a FITC on its 5′ portion or 3′ portion. In some embodiments, a single stranded reporter oligonucleotide comprises an extension blocking group on its 5′ portion or 3′ portion.

In some aspects, the disclosure provides a method for detecting target nucleic acids in a biological sample, the method comprising contacting an amplification mixture as described herein with a biological sample; performing an isothermal amplification reaction on the mixture to produce an amplification mixture; contacting the amplification mixture to a lateral flow assay (LFA) device under conditions under which the oligonucleotide strand displacement probe in the amplification mixture produces a first detectable signal when a target nucleotide is absent from the biological sample and/or a second detectable signal when a control nucleic acid is present in the biological sample; identifying the presence of the target nucleic acid in the biological sample based upon detecting the absence of a first detectable signal and the presence of a second detectable signal, or identifying the absence of the target nucleic acid in the biological sample based on detecting the presence of the first detectable signal and the presence of the second detectable signal.

In some aspects, the disclosure provides a method for detecting target nucleic acids in a biological sample, the method comprising contacting the amplification mixture as described herein with a biological sample; performing an isothermal amplification reaction on the mixture to produce an amplification mixture; contacting the amplification mixture to a lateral flow assay (LFA) device under conditions under which the single stranded reporter oligonucleotide in the amplification mixture produces a first detectable signal when a target nucleotide is present from the biological sample or a second detectable signal when the target nucleic acid is absent from the biological sample; identifying the presence of the target nucleic acid in the biological sample based upon detecting the presence of a first detectable signal and the absence of a second detectable signal, or identifying the absence of the target nucleic acid in the biological sample based on detecting the absence of the first detectable signal and the presence of the second detectable signal.

In some embodiments, a biological sample comprises blood, saliva, mucous, urine, feces, cerebrospinal fluid (CSF), or tissue.

In some embodiments, a target nucleic acid is derived from a pathogen. In some embodiments, a pathogen is a viral, bacterial, fungal, parasitic, or protozoan pathogen. In some embodiments, a viral pathogen is SARS-CoV-2.

In some embodiments, methods described herein further comprise a step of detecting the presence of one or more control nucleic acids in a biological sample. In some embodiments, one or more control nucleic acids are derived from a human.

In some aspects, the disclosure provides an isolated nucleic acid comprising or consisting of the sequence set forth in any one of SEQ ID NOs: 1-103.

### BRIEF DESCRIPTION OF THE DRAWINGS

FIG. 1 shows, according to some embodiments, a detection component comprising a conjugate pad containing immobilized carbon particles coated with a first or a second affinity agent (e.g., streptavidin, mouse serum, etc.), a first test line comprising a binding agent specific for a first detectable label (e.g., first hapten), a second test line comprising a binding agent specific for a second detectable label (e.g., second hapten), a control line comprising a binding agent specific for a third detectable label (e.g., a second affinity agent), and optionally an absorbent pad.

FIG. 2 shows, according to some embodiments, a rapid SARS-CoV-2 test. A single-tube assay is initiated when a human sample is introduced into the assay tube and concluded by the direct addition of the product to a lateral flow “reader,” which results in the production of 1 or more visible bands. Unique banding patterns correspond to positive, negative and invalid results.

FIG. 3 shows representative data indicating that Reverse transcription loop-mediated isothermal amplification (RT-LAMP) products directly applied to lateral flow immunoassay strips generate dim visible bands unless they are previously diluted.

FIG. 4 shows representative data indicating that multiplexed RT-LAMP products directly applied to lateral flow immunoassay strips generate dim visible bands unless they are previously diluted.

FIG. 5 shows representative data for the effect of dilution ratio on the development of lateral flow band intensity of RT-LAMP products. Dilution of LAMP products prior to addition to lateral flow device increases band intensity. RT-LAMP assay buffer is a high performing diluent that increases lateral flow strip band signal. Water lacks surfactant and is not a good diluent.

FIG. 6 shows representative data indicating that RT-LAMP product containing no detectable labels is a suitable diluent for a labeled RT-LAMP reaction.

FIG. 7 shows representative data indicating that reduction in hapten—(e.g., FITC) and biotin-labeled nucleic acid results in increased lateral flow band brightness.

FIG. 8 shows representative data indicating that addition of excess biotinylated primers (oligonucleotides) dims lateral flow signal.

FIG. 9 shows representative data indicating that addition of excess FITC-labeled primers dims lateral flow signal.

FIG. 10 shows representative data indicating that RT-LAMP reactions performed using attenuated labeled LoopB and LoopF oligonucleotide primers generate brighter, more visible bands when directly applied to lateral flow strips with no intervening steps or dilution.

FIG. 11 shows representative data indicating that decreasing the concentrations of labeled LoopF and LoopB oligonucleotides results in slower RT-LAMP amplification reactions.

FIG. 12 shows representative data indicating that decreasing the concentrations of labeled LoopF and LoopB oligonucleotides results in slower RT-LAMP amplification reactions.

FIG. 13 shows representative data indicating the more the concentrations of labeled LoopF and LoopB oligonucleotides are reduced, the slower the resulting RT-LAMP amplification reaction.

FIG. 14 shows representative data indicating that attenuating the labeled loop concentration in an RT-LAMP reaction increases brightness of bands evolved on a lateral flow strip when the RT-LAMP products are directly applied with no dilution step.

FIG. 15 shows representative data indicating that addition of unlabeled loop oligonucleotide primers to a reaction buffer comprising attenuated loop oligonucleotide primers accelerates the RT-LAMP reaction, particularly in the presence of higher template concentrations.

FIG. 16 shows representative data indicating that addition of unlabeled loop oligonucleotide primers to a reaction buffer comprising attenuated loop oligonucleotide primers does result in decreased lateral flow band brightness.

FIG. 17 shows representative data indicating the closer a reaction enters the exponential amplification phase to the conclusion of a RT-LAMP reaction and application of the products to a lateral flow strip, the brighter the evolved band intensity signal.

FIG. 18 shows representative data indicating that labeling Forward Internal Primer (FIP) and LoopF primers with chemical moieties detectable on lateral flow results in superior band brightness relative to other labeled primer pairs.

FIG. 19 shows representative data indicating that labeling FIP and LoopF primers with chemical moieties detectable on lateral flow, in some embodiments, results in false positive signals for certain primer sets.

FIG. 20 shows representative data indicating that reducing FIP/Backward Internal Primer (BIP) concentrations from 1600 nM (primer set A) down to 400 nM (primer set C) increases lateral flow band brightness for reactions that are undiluted and applied directly to the strip.

FIGS. 21A-21C show signal detection using strand displacement probes. FIG. 21A shows dual-labeled oligonucleotide strand displacement (OSD) probes may be used to report amplification on lateral flow strips. FIGS. 21B-21C shows that, in some embodiments, the signal is inverted such that samples with template concentrations less than the limit of detection (e.g., negative samples) produce a visible band on lateral flow and samples with sufficient template for amplification (e.g., positive samples) exhibit an absence of a visible band.

FIGS. 22A-22B show schematics depicting embodiments of oligonucleotide strand displacement probes. FIG. 22A shows one embodiment of a labeled oligonucleotide displacement probe. FIG. 22B shows oligonucleotide strand displacement probe cascades that invert signals for a visible “ON” or band shift upon amplification.

FIGS. 23A-23B show embodiments of mediator displacement probes. FIG. 23A shows conventional signal transduction by Mediator Displacement Probes (MDP). In some embodiments, modifications for fluorescent molecules (e.g., a quencher) are optional. In the illustration, a target to a forward loop is shown, but the MDP can be equally applied to target a reverse loop. FIG. 23B shows modification of mediator displacement probes for the generation of an LFA analyte by decoupling LAMP from LFA detection. This is achieved by 3 probes: one probe targeting the LAMP amplicon (e.g., ‘T_medC’ or ‘target+medC’), a second probe containing a first detectable label called ‘Universal Reporter’ (UR), and a third probe called ‘Med’ or ‘Mediator’ probe which is complementary to both T_medC and the Universal Reporter and which contains a second detectable label. In the “OFF” state Med probes are annealed to T_medC, which results in no (or a minimal amount of) dually-labelled molecules for LFA detection. During LAMP amplification, T_medC is incorporated and Med probe is released after subsequent strand-displacement, binding to the universal reporter. In embodiments in which the 3′ end of the Med probe is free, it will lead to polymerization using Universal Reporter as template further stabilizing the dually-labelled molecule.

FIG. 24 shows representative data indicating that blocking the 3′-end of a Med probe reduces background LFA signal.

FIG. 25 shows representative data for detection of Ex2b (RNAse P) amplicon using a Ex2b_LF/Med2-BIO[BK] probe. Ex2b_LF_Med2 probe and a Med2-BIO blocked probe were titrated in the presence of Ex2b primer set (minus LF) amplification and As1e (Sars-CoV-2) amplification. The TmedC/Med ratio was kept constant at 2:1 ratio. UR-DIG was also constant at 10 nM. Data indicate that near the 50-100 nM Med2 range, specific signal generation was produced on LFA strips.

### DETAILED DESCRIPTION

The disclosure relates, in some aspects, to compositions and methods for amplifying and/or detecting target analytes (e.g., nucleic acids) in a sample. The disclosure is based, in part, on methods that reduce the amount of one or more labeled primers in an amplification reaction but still produce high contrast, visible, colored bands when bound to certain immunoassay devices (e.g., lateral flow immunoassays). The disclosure is also based, in part, on labeled oligonucleotide strand displacement probes which report the presence or absence of a target nucleic acid in a sample following an isothermal amplification reaction. In some embodiments, compositions and methods described herein represent an improvement over currently available amplification and detection methods because they allow for direct application of amplicons (e.g., solutions comprising amplicons) to immunoassay devices without any intervening fluid transfer or dilution steps.

In some aspects, the disclosure describes compositions and methods for the direct application of reacted nucleic acid amplification (NAA) mixes to lateral flow strips without any intervening dilution or sample preparation steps. In some embodiments, the disclosed methods incorporate secondary nucleic acid reactions alongside the primary NAA reaction responsible for amplifying the diagnostic target sequence. These secondary nucleic acid reactions result in the formation of controllable amounts of secondary products of desired size, structure and chemical labelling, position, and/or stoichiometry, which function as ideal lateral flow assay (LFA) analytes according to these properties. In some embodiments, the secondary nucleic acid reactions are caused by the presence of one or more oligonucleotide strand displacement probes (OSDs) in an isothermal amplification reaction, such as a LAMP reaction.

### Oligonucleotide Strand Displacement Probes

Aspects of the disclosure relate to the recognition that inclusion of certain oligonucleotide strand displacement (OSD) probes in isothermal amplification reactions (e.g., LAMP) allows for direct application of the resulting amplicon mixtures to immunoassay devices without any intervening fluid transfer or dilution steps. An “oligonucleotide strand displacement probe” generally refers to a modified polynucleotide primer comprising a region of complementarity with one or more target nucleotides that is capable of displacing pre-hybridized strands of target nucleic acid amplicons (e.g., amplicons generated by LAMP). Oligonucleotide stand-displacement probes are generally known, for example as described by Jiang et al., Angew Chem Int Ed Engl. 2014 Feb. 10; 53(7): 1845-1848; Phillips et al., Anal Chem. 2018 Jun. 5; 90(11): 6580-6586; and Bhadra et al. (2020) https://doi.org/10.1101/2020.04.13.039941.

In some aspects, the disclosure provides an oligonucleotide strand displacement probe comprising a first single stranded polynucleotide having a 5′ portion and a 3′ portion, wherein the 5′ portion comprises a first hapten and the 3′ portion comprises an extension blocking group; and a second single stranded polynucleotide comprising a 5′ portion and a 3′ portion, wherein the 5′ portion comprises a biotin. The second single stranded polynucleotide is hybridized to the first single stranded polynucleotide to form a duplex region and a single stranded toehold region comprising the 3′ portion of the first single stranded polynucleotide.

In some embodiments, a first single stranded polynucleotide of an OSD probe ranges from about 10 to about 50 nucleotides in length. In some embodiments, the first single stranded polynucleotide is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a second single stranded polynucleotide of an OSD probe ranges from about 10 to about 50 nucleotides in length. In some embodiments, the second single stranded polynucleotide is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an OSD probe forms a duplex molecule. In some embodiments, the duplex region of an OSD probe ranges from about 5 nucleotides to about 40 nucleotides in length. In some embodiments, the duplex region is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a duplex region comprises a blunt end. The blunt end may be the 5′ end of the duplex or the 3′ end of the duplex (e.g., relative to the sense strand of the OSD probe). In some embodiments, the 5′ end or 3′ end of an OSD comprises a blocking group, as described herein.

A “toehold region” is a single stranded portion of an OSD probe that comprises the region of complementarity with the target nucleic acid. In some embodiments, a single stranded toehold region ranges from about 5 to about 45 nucleotides in length. In some embodiments, the toehold region is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length.

A OSD probe typically comprises two or more modifications. In some embodiments, an OSD probe comprises a first modification on the 5′ end (relative to the sense strand) and a second modification on the 3′ end (relative to the sense strand). In some embodiments, the first modification comprises a first hapten. In some embodiments, a first hapten comprises FITC. In some embodiments, a second modification comprises an extension blocking group. In some embodiments, an extension blocking group comprises a dehydroxylated 3′ nucleotide, 3′ dideoxycytosine (ddC), 3′ inverted deoxythymidine (dT), 3′ propyl (C3) spacer, 3′ amino modification, 3′ phosphoryl modification, or 3′ biotin modification.

The disclosure also relates to additional reporter molecules which may be used with OSD probes in isothermal amplification reactions. In some embodiments, a reporter molecule comprises a single stranded reporter oligonucleotide. In some embodiments, a single stranded reporter oligonucleotide comprises a FITC on its 5′ portion or 3′ portion. In some embodiments, a single stranded reporter oligonucleotide comprises an extension blocking group on its 5′ portion or 3′ portion. The length of a single stranded reporter oligonucleotide may vary. In some embodiments, a single stranded reporter oligonucleotide ranges from about 5 to 50 nucleotides in length, 10 to 30 nucleotides in length, or 8 to 25 nucleotides in length. In some embodiments, a single stranded reporter oligonucleotide reporter is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a single stranded reporter molecule forms a hairpin structure (e.g., a stem-loop structure). In some embodiments, a reporter molecule comprises a double-stranded reporter oligonucleotide.

An isothermal amplification reaction (e.g., a LAMP reaction) may comprise one or more OSDs and one or more additional reporter molecules. In some embodiments, an isothermal amplification reaction comprises 2, 3, 4, 5, 6, 7, 8, or more OSDs. In some embodiments, an isothermal amplification reaction comprises 2, 3, 4, 5, 6, 7, 8, or more additional reporter molecules. In some embodiments, an isothermal amplification reaction comprises 2, 3, 4, 5, 6, 7, 8, or more OSDs and 2, 3, 4, 5, 6, 7, 8, or more additional reporter molecules.

### Mediator Displacement Probes

In some embodiments, the one or more OSDs comprise one or more mediator displacement probes (MDPs). As used herein, the term “mediator displacement probes” or “MDPs” refers to a probe or probe set (e.g., two or more probes that function together in secondary reactions during an isothermal amplification reaction), wherein at least one of the probes comprises a hemi-duplex primer that targets a loop on an RT-LAMP amplicon. The primer comprises a target sequence specific portion that is annealed to a single stranded mediator oligonucleotide (referred to as a ‘Med’ oligonucleotide). During amplification, a strand displacing polymerase drives release of the annealed Med oligonucleotide by extension and strand-displacement. The released strand hybridizes to a single stranded nucleic acid ‘Universal Reporter’ and primes its extension into a longer dual labeled duplex secondary product. Mediator displacement probes are generally known, for example as described by Becherer et al. Anal. Chem. 2018, 90, 4741-4748, the entire contents of which are incorporated herein by reference. Universal Reporter (UR) probes are also known, for example as described by Lehnert et al. Anal. Methods, 2018, 10, 3444, the entire contents of which are incorporated herein by reference.

The disclosure is based, in part, on mediator displacement probes comprising one or more modifications relative to previously used MDPs, and which reduce background signal noise during lateral flow assay (LFA) detection. In some embodiments, the one or more modifications comprise an extension blocking group on the 3′ end of the Universal Reporter of a MDP probe set. Examples of extension blocking groups include but are not limited to dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, the one or more modifications comprises a detectable labels for LFA, for example, biotin, FAM, or DIG. The modifications can be added either to the 5′ of a Med probe, and/or to a 3′ of a Universal Reporter.

In some embodiments, the disclosure provides a mediator probe set comprising: a first single stranded oligonucleotide Med probe comprising a detectable label, and a second hemi-duplex oligonucleotide probe comprising a single stranded target sequence-specific portion contiguously linked (e.g., sharing the same phosphate-based backbone) with a MedC oligonucleotide that is complementary to the Med probe. In some embodiments, the Med probe is annealed (e.g., hybridized) to the MedC portion of the second hemi-duplex oligonucleotide probe. In some embodiments, the Med probe further comprises an extension blocking group on its 3′ end. In some embodiments, the mediator probe set further comprises a Universal Reporter probe comprising an oligonucleotide sequence that is complementary to the Med probe. In some embodiments, the Universal Reporter probe comprises a detectable label. In some embodiments, the detectable label is positioned on the 3′ end of the Universal Reporter. In some embodiments, the Universal Reporter probe further comprises an extension blocking group on its 5′ end.

### Biological Samples

Aspects of the disclosure relate to methods for detecting one or more target nucleotides in a biological sample. In some embodiments, biological sample is obtained from a subject (e.g., a human subject, an animal subject). Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, a biological sample comprises blood, saliva, mucous, urine, feces, cerebrospinal fluid (CSF), or tissue.

In some embodiments, the biological sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds. In some embodiments, the biological sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020) an amount that is detectable by any one of the methods described herein. In some embodiments, methods described herein are capable of detecting a concentration of a target nucleic acid (e.g., SARS-Cov-2 RNA) in a biological sample that is less than 5 fM.

In some embodiments, a biological sample (e.g., saliva sample) is deposited directly into a reaction tube. In some embodiments, the concentration of a target nucleic acid molecule (e.g., SARS-CoV-2 RNA) in the biological sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the biological sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of a target nucleic acid molecule (e.g., SARS-CoV-2 RNA) in the biological sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The biological sample, in some embodiments, is collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

A subject may be any mammal, for example a human, non-human primate (e.g., monkey, chimpanzee, ape, etc.), dog, cat, pig, horse, hamster, guinea pig, rat, mouse, etc. In some embodiments, a subject is a human. In some embodiments, a subject is an adult human (e.g., a human older than 16 years of age, 18 years of age, etc.). In some embodiments, a subject is a child (e.g., a pediatric subject), for example a subject that is less than 18 years of age, 16 years of age, etc. In some embodiments, a subject is an infant, for example a subject less than one year of age.

### Nucleic Acids

The disclosure relates, in some aspects, to nucleic acids and nucleic acid sequences. A “nucleic acid” sequence refers to a DNA or RNA (or a sequence encoded by DNA or RNA). In some embodiments, a nucleic acid is isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which can be manipulated by recombinant DNA techniques well known in the art. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In some embodiments, a nucleic acid or isolated nucleic acid is a referred to as a “polynucleotide” or “oligonucleotide”. The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-O-methyl-RNA, or combinations thereof.

The length of a polynucleotide (or each strand of a double stranded, hemi-duplex, or duplex molecule) may vary. In some embodiments, a polynucleotide ranges from about 2 to 10, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 2000, 2 to 5000, 2 to 10,000, 2 to 50,000, 2 to 500,000, or 2 to 1,000,000 nucleotides in length. In some embodiments, a polynucleotide is more than 1,000,000 nucleotides in length (e.g., longer than a megabase).

A nucleic acid (e.g., a polynucleotide) may be single stranded or double stranded. In some embodiments, a single stranded polynucleotide comprises a sequence of polynucleotides connected by a contiguous backbone. In some embodiments, a single stranded polynucleotide comprises a 5′ portion (end or terminus) and a 3′ portion (end or terminus). A single stranded polynucleotide may be a sense strand or an antisense strand.

In some embodiments, a nucleic acid (e.g., polynucleotide) double stranded. A double stranded polynucleotide comprises a first (e.g., “sense”) polynucleotide strand that is hybridized to a second polynucleotide (“antisense”) strand via hydrogen bonding between the nucleobases of each strand along a region of complementarity between the two strands. Each strand of a double stranded polynucleotide comprises a 5′ portion and a 3′ portion.

As used herein, the term “region of complementarity” refers to a region of a nucleic acid (e.g., polynucleotide) that is substantially complementary (e.g., forms Watson-Crick base pairs) to another nucleic acid sequence, for example a target nucleic acid sequence, control nucleic acid sequence, or a corresponding antisense strand (e.g., in the case of a double stranded or duplex nucleic acid). Two nucleic acids, for example a sense strand and antisense strand of a double stranded polynucleotide, may comprise a region of complementarity that ranges from about 70% to about 100% complementarity. In some embodiments, two polynucleotides share a region of complementarity that is about 50%, 60%, 70%, 80%, 90%, 95%, 99%, or 100% complementary. In some embodiments, a region of complementarity between two nucleic acids comprises at least 1, 2, 3, 4, 5, 6, 7, 8, 9, 10, 15, 20, 25, 50, 100, 200, or 500 mismatched nucleotide bases. In some embodiments, a region of complementarity between two nucleic acids comprises between 1 and 5, 2 and 10, 5 and 15, 10 and 50, 30 and 100, or 75 and 500 mismatched nucleotide bases. Mismatched nucleotide bases within a region of complementarity may result in the resulting complex comprising one or more bulges or loops.

In some embodiments, a single stranded polynucleotide or a double stranded polynucleotide forms a duplex region. A “duplex” refers to a stable complex formed by fully or partially complementary polynucleotides that undergo Watson-Crick type base pairing along a region of complementarity. For example, a sense strand and its reverse-complement antisense strand form a duplex molecule when hybridized (e.g., annealed) together. In another example, a single stranded polynucleotide forms a duplex molecule when two portions of the polynucleotide self-hybridize to form a stem-loop (e.g., hairpin) structure.

A duplex molecule may comprise one or two blunt ends. A “blunt end” refers to a double stranded polynucleotide (e.g., a duplex molecule) where the one end of the first (sense) polynucleotide strand and one end of the second (e.g., antisense) strand terminate at the same nucleotide position such that no overhang is formed. A blunt end may be formed at either the 5′ end or the 3′ end (e.g., with respect to the sense strand) of a duplex molecule. In some embodiments, a duplex molecule comprises two blunt ends. In some embodiments, a duplex molecule comprises one blunt end and one overhang. An “overhang” refers to a single stranded region of a duplex molecule formed by asymmetric hybridization of the first (e.g., sense) and second (e.g., antisense) strands of a duplex. Asymmetric hybridization may result from a difference in the lengths of two polynucleotides forming the duplex (e.g., one polynucleotide is longer than another) or from the two polynucleotide strands sharing a region of complementarity that does not encompass the entire length of one (or both) of the polynucleotides. Thus, in some embodiments, a duplex molecule comprises two overhangs.

In some embodiments, a double stranded polynucleotide having an overhang is a hemi-duplex molecule. As used herein, “hemi-duplex” refers to a double stranded polynucleotide molecule in which one polynucleotide strand is longer than the other polynucleotide strand, such that when the strands are hybridized (or annealed) to one another, the resulting double stranded molecule comprises a single stranded portion and a double stranded portion. In some embodiments, the single stranded portion of a hemi-duplex molecule is referred to as a “toehold” portion of the hemi-duplex.

The length of an overhang may vary. In some embodiments, an overhang ranges from between 1 and 100 nucleotides in length. In some embodiments, an overhang ranges from between 1 and 5 nucleotides in length. In some embodiments, an overhang ranges from between 2 and 10, 5 and 15, 10 and 25, or 20 and 100 nucleotides in length. In some embodiments, an overhang is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a nucleic acid (e.g., polynucleotide) is a primer. As used herein, a “primer” refers to a polynucleotide that is capable of selectively binding (e.g., hybridizing or annealing) to a nucleic acid template and allows the synthesis of a sequence complementary to the corresponding polynucleotide template. A nucleic acid template may be a target nucleic acid or control nucleic acid. In some embodiments, a nucleic acid template comprises a region of complementarity with one or more primers. Generally, a primer ranges in size from about 10 to 100 nucleotides, and functions as a point of initiation for template-directed, polymerase mediated synthesis of a polynucleotide complementary to the template. In some embodiments, a primer is specific for a target nucleic acid. In some embodiments, a primer is specific for a control nucleic acid. A primer that is “specific” for a template binds (e.g., hybridizes) to that template with higher affinity than any other nucleic acid. In some embodiments, a primer that is “specific” for a template does not bind to any other nucleic acids present in a sample along with the template. A “pair of primers” or “primer pair” refers to two primers that bind to different regions (or portions) of the same template (e.g., the same target nucleic acid).

In some embodiments, a primer is an outer primer. An “outer primer” refers to a primer that binds at or near a 5′ end (e.g., at or near the 5′ terminal nucleotide) of a template sequence (e.g., a target nucleic acid, control nucleic acid, etc.), or at or near a 3′ end (e.g., at or near the 3′ terminal nucleotide) of a template sequence (e.g., a target nucleic acid, control nucleic acid, etc.) and is capable of displacing, or un-hybridizing, the two strands of a duplex upon hybridization of the primer to one of the duplex strands. In some embodiments, an outer primer binds to a terminal nucleotide, such as a 5′ terminal nucleotide or a 3′ terminal nucleotide of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 5′ end (terminal nucleotide) of a template. In some embodiments, an outer primer binds within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of the 3′ end (terminal nucleotide) of a template. In some embodiments, an outer primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, a primer is an internal primer. An “internal primer” refers to a primer that binds internally to an outer primer (e.g., downstream or 3′ relative to a 5′ outer primer, or upstream or 5′ relative to a 3′ outer primer). In some embodiments, internal primers are referred to as “nested primers”. In some embodiments, an internal primer binds to nucleic acid sequence that is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides of an outer primer. In some embodiments, an internal primer binds more than 50 nucleotides away from an outer primer, for example 60, 70, 80, 90, 100, 150, 200, or more nucleotides upstream or downstream of an outer primer. In some embodiments, an internal primer specifically hybridizes to a target nucleic acid. In some embodiments, an internal primer specifically hybridizes to a control nucleic acid. In some embodiments, an internal primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, an internal primer is a loop primer. A “loop primer” refers to a primer that binds to a single stranded duplex (e.g., loop) region of a template nucleic acid that is formed during Loop-mediated isothermal amplification (LAMP). In some embodiments, loop primers accelerate amplification (e.g., decrease the reaction time) during LAMP. In some embodiments, a loop primer binds to a region of a template (e.g., target nucleic acid) that is downstream of (e.g., 3′ relative to) an outer primer or another internal primer. In some embodiments, a loop primer binds to a region of a template (e.g., target nucleic acid) that is upstream of (e.g., 5′ relative to) an outer primer or another internal primer. In some embodiments, a loop primer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

A nucleic acid may be unmodified or modified. A modified nucleotide may comprise one or more modified nucleic acid bases and/or a modified nucleic acid backbone. In some embodiments, a modified nucleic acid comprises one or more nucleotide analogs. The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH.sub.2, NHR, NR.sub.2, COOR, or, wherein R is substituted or unsubstituted C.sub.1-C.sub.6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

In some embodiments, a nucleic acid is modified by conjugation to one or more biological or chemical moieties. Examples of moieties used for modifying nucleic acids include fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof. In some embodiments, a nucleic acid (e.g., a primer) comprises one or more modifications. In some embodiments, a modified nucleic acid comprises a hapten. In some embodiments, the hapten is FITC. In some embodiments, the hapten is digoxigenin (DIG). In some embodiments, the hapten is biotin. In some embodiments, a modified nucleic acid comprises an extension blocking group. Examples of extension blocking groups include but are not limited to dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, a modified nucleic acid comprises both a hapten and an extension blocking group.

### Target Nucleic Acids

Methods and compositions described by the disclosure may be used, in some embodiments, to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G mutation (i.e., a mutation of the 614^{th} amino acid from aspartic acid (D) to glycine (G)) in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, SARS-CoV-2 D614G, an influenza type A virus, and/or an influenza type B virus). In some embodiments, a diagnostic device comprises a lateral flow assay strip comprising four or more test lines (e.g., test lines configured to detect SARS-CoV-2, SARS-CoV-2 D614G, an influenza type A virus, and/or an influenza type B virus).

In some embodiments, a diagnostic device, system, or method is configured to detect at least 1, at least 2, at least 3, at least 4, at least 5, at least 6, at least 7, at least 8, at least 9, or at least 10 target nucleic acid sequences. In some embodiments, the diagnostic device is configured to detect 1 to 2 target nucleic acid sequences, 1 to 5 target nucleic acid sequences, 1 to 8 target nucleic acid sequences, 1 to 10 target nucleic acid sequences, 2 to 5 target nucleic acid sequences, 2 to 8 target nucleic acid sequences, 2 to 10 target nucleic acid sequences, 5 to 8 target nucleic acid sequences, 5 to 10 target nucleic acid sequences, or 8 to 10 target nucleic acid sequences. Each target nucleic acid sequence may independently be a nucleic acid of a pathogen (e.g., a viral, bacterial, fungal, protozoan, or parasitic pathogen) and/or a cancer cell.

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or SARS-CoV-2 D614G. In certain embodiments, the viral pathogen is an influenza virus. The influenza virus may be an influenza A virus (e.g., HIN1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, the diagnostic devices, systems, and methods are configured to detect a target nucleic acid sequence of a bacterium (e.g., a bacterial pathogen). The bacterium may be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the diagnostic devices, systems, and methods are configured to detect a target nucleic acid sequence of a fungus (e.g., a fungal pathogen). Examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the diagnostic devices, systems, and methods are configured to detect a target nucleic acid sequence of one or more protozoa (e.g., a protozoan pathogen). Examples of protozoan pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

In some embodiments, the diagnostic devices, systems, and methods are configured to detect a target nucleic acid sequence of a parasite (e.g., a parasitic pathogen). Examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, Schistosoma (liver fluke), Loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the diagnostic devices, systems, and methods are configured to detect a target nucleic acid sequence of a cancer cell. Cancer cells have unique mutations found in tumor cells and absent in normal cells. For example, the diagnostic devices, systems, and methods may be configured to detect a target nucleic acid sequence encoding a cancer neoantigen, a tumor-associated antigen (TAA), and/or a tumor-specific antigen (TSA). Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 0-Catenin, CDK4, Mum-1, p16, TAGE, PSMA, PSCA, CT7, telomerase, 43-9F, 5T4, 791Tgp72, alpha-fetoprotein, 3-HCG, BCA225, BTAA, CA 125, CA 15-3 (CA 27.29BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein\cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some embodiments, the diagnostic devices, systems, and methods are configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA1 and/or BRCA2 genes. In some instances, the diagnostic devices, systems, and methods are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the diagnostic devices, systems, and methods are configured to detect a target nucleic acid sequence associated with a genetic disorder. Non-limiting examples of genetic disorders include hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESC02, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the diagnostic devices, systems, and methods are configured to detect a target nucleic acid sequence of an animal pathogen. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

The diagnostic devices, systems, and methods described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic devices, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus. Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the diagnostic devices, systems, or methods described herein, one can determine whether the sample contains the one or more bacterial toxins. In some embodiments, the diagnostic devices, systems, or methods may be operated or conducted during the food production process to ensure food safety prior to consumption.

In some embodiments, the diagnostic devices, systems, and methods described herein may be used to test samples of soil, building materials (e.g., drywall, ceiling tiles, wall board, fabrics, wallpaper, and floor coverings), air filters, environmental swabs, or any other sample. In certain embodiments, the diagnostic devices, systems, and methods may be used to detect one or more toxins, as described above. In certain instances, the diagnostic devices, systems, and methods may be used to analyze ammonia- and methane-oxidizing bacteria, fungi or other biological elements of a soil sample. Such information can be useful, for example, in predicting agricultural yields and in guiding crop planting decisions.

### Control Nucleic Acid Sequences

In some embodiments, methods and compositions described herein comprise (or use) primers designed to amplify a human or animal nucleic acid that is not associated with a target nucleic acid from a pathogen, a cancer cell, or a contaminant. In some such embodiments, the human or animal nucleic acid may act as a control and is referred to as a “control nucleic acid”. For example, successful amplification and detection of a control nucleic acid may indicate that a sample was properly collected, and the diagnostic test was properly run (e.g., an amplification reaction was successful).

A control nucleic acid is typically a gene or portion of a gene that is widely expressed and/or expressed at a high level in a control organism (e.g., a human or other mammal). Such genes are typically referred to as “housekeeping genes”. Examples of housekeeping genes include but are not limited to GAPDH, B2M, ACTB, POLR2A, UBC, PPIA, HPRT1, GUSB, TBP, and H3F3A. In some embodiments, a control nucleic acid encodes a gene (or portion of a gene) selected from GAPDH, B2M, ACTB, POLR2A, UBC, PPIA, HPRT1, GUSB, TBP, H3F3A, POLR2A, RPLPO, L19, B2M, RPS17, ALAS1, CD74, RNase P, CK18, HMBS, IP08, PGK1, YWHAZ, and STATH.

In some embodiments, a diagnostic device, system, or method is configured to detect at least 1, at least 2, at least 3, at least 4, at least 5, at least 6, at least 7, at least 8, at least 9, or at least 10 control nucleic acid sequences. In some embodiments, the diagnostic device is configured to detect 1 to 2 control nucleic acid sequences, 1 to 5 control nucleic acid sequences, 1 to 8 control nucleic acid sequences, 1 to 10 control nucleic acid sequences, 2 to 5 control nucleic acid sequences, 2 to 8 control nucleic acid sequences, 2 to 10 control nucleic acid sequences, 5 to 8 control nucleic acid sequences, 5 to 10 control nucleic acid sequences, or 8 to 10 control nucleic acid sequences. Each control nucleic acid sequence may independently be a nucleic acid of a human or another animal (e.g., mammal).

### Lysis of Sample

A biological sample may be lysed prior to (or while) performing an amplification reaction (e.g., an isothermal amplification reaction, such as RT-LAMP). In some embodiments, lysis of a biological sample is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulose, protease, and glycanase.

In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, an amplification buffer described herein comprises one or more detergents. In some embodiments, the one or more detergents comprises Tween.

In some cases, at least one of the one or more lysis reagents is in solid form (e.g., lyophilized, dried, crystallized, air jetted). In some cases, all of the one or more lysis reagents are in solid form (e.g., lyophilized, dried, crystallized, air jetted). In certain embodiments, one or more lysis reagents are in the form of a lysis pellet or tablet. The lysis pellet or tablet may comprise any lysis reagent described herein. In certain embodiments, the lysis pellet or tablet may comprise one or more additional reagents (e.g., reagents to reduce or eliminate cross contamination). In a particular, non-limiting embodiment, a lysis pellet or tablet comprises Thermolabile Uracil-DNA Glycosylase (UDG) (e.g., at a concentration of about 0.02 U/μL) and murine RNAse inhibitor (e.g., at a concentration of about 1 U/μL).

In some embodiments, the lysis pellet or tablet is shelf stable for a relatively long period of time. In certain embodiments, the lysis pellet or tablet is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the lysis pellet or tablet is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the lysis pellet or tablet is thermostabilized and is stable across a wide range of temperatures. In some embodiments, the lysis pellet or tablet is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the lysis pellet or tablet is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.). In some embodiments, chemical lysis is performed at a temperature in a range from 20° C. to 25° C., 20° C. to 30° C., 20° C. to 37° C., 20° C. to 50° C., 20° C. to 60° C., 20° C. to 65° C., 20° C. to 70° C., 20° C. to 80° C., 20° C. to 90° C., 25° C. to 30° C., 25° C. to 37° C., 25° C. to 50° C., 25° C. to 60° C., 25° C. to 65° C., 25° C. to 70° C., 25° C. to 80° C., 25° C. to 90° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period. In certain instances, the first temperature is at least 37° C., at least 50° C., at least 60° C., at least 65° C., at least 70° C., at least 80° C., or at least 90° C. In certain instances, the first temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In some embodiments, a lysis heating protocol comprises heating the sample at a second temperature for a second time period. In certain instances, the second temperature is at least 37° C., at least 50° C., at least 60° C., at least 65° C., at least 70° C., at least 80° C., or at least 90° C. In certain instances, the second temperature is in a range from 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the second time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes. In a particular, non-limiting embodiment, the first temperature is in a range from 37° C. to 50° C. (e.g., about 37° C.) and the first time period is in a range from 1 minute to 5 minutes (e.g., about 3 minutes), and the second temperature is in a range from 60° C. to 70° C. (e.g., about 65° C.) and the second time period is in a range from 5 minutes to 15 minutes (e.g., about 10 minutes). In some embodiments, a lysis heating protocol may comprise heating a sample at one or more additional temperatures for one or more additional time periods.

### Methods of Amplifying Nucleic Acids

Aspects of the disclosure relate to methods and compositions for detecting target nucleic acids and/or control nucleic acids in a biological sample. In some embodiments, the methods comprise a step of performing an isothermal amplification reaction on a biological sample in order to amplify a target nucleic acid and/or a control nucleic acid.

The disclosure is based, in part, on amplification buffer solutions comprising one or more of the following components: biotinylated oligonucleotides, a target nucleic acid-specific primer comprising a first hapten, and a control nucleic acid-specific primer comprising a second hapten different from the first hapten. In some embodiments, the buffer comprises a total concentration of biotinylated oligonucleotides that is less than 200 nM, a total concentration of first hapten less than or equal to 25 nM, and a total concentration of second hapten that is less than or equal to 100 nM. In some embodiments, the result of performing an amplification reaction using such a buffer is production of a mixture of amplification products that can be directly added to a detection device, for example a lateral flow assay (LFA) device, and which results in visible, colored bands. This is surprising because it was previously thought that amplification mixtures needed to be diluted prior to addition to a detection device in order to produce brightly visible bands.

Thus, in some embodiments, following lysis, one or more target nucleic acids (e.g., a nucleic acid of a target pathogen) and/or one or more control nucleic acids may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, reverse transcription is performed by exposing lysate (or nucleic acids within a lysate) to one or more reverse transcription reagents. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid.

In some embodiments, a reverse transcriptase used for an isothermal amplification reaction (e.g., RT-LAMP) is selected from the group consisting of HIV-1 reverse transcriptase, Moloney murine leukemia virus (M-MLV) reverse transcriptase, and avian myeloblastosis virus (AMV) reverse transcriptase.

In some embodiments, DNA may be amplified according to any nucleic acid amplification method known in the art. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction.

In some embodiments, the isothermal amplification methods described below include a modified nucleotide, for example, deoxyuridine triphosphate (dUTP), during amplification. In such embodiments, a subsequent test may comprise a uracil-DNA glycosylase (UDG) treatment prior to the amplification step, followed by a heat inactivation step (e.g., 95° C. for 5 minutes) (Hsieh et al., Chem Comm, 2014, 50: 3747-3749). In some embodiments, the heat inactivation step may correspond to a thermal lysis step.

Without wishing to be bound by a particular theory, it is thought that the addition of dUTP during the amplification process will reduce or eliminate potential contamination between samples. In the absence of adding dUTP and UDG, amplicons may aerosolize and contaminate future tests, potentially result in false positive test results. The use of UDG (thermolabile Uracil DNA glycosylase) generally prevents carryover contamination by specifically degrading products have already been amplified (i.e., amplicons), leaving the unamplified (new) sample untouched and ready for amplification. Using this method, tests may be performed sequentially in the same tube and/or in the same area.

In some cases, at least one of the one or more amplification reagents is in solid form (e.g., lyophilized, dried, crystallized, air jetted). In some cases, all of the one or more amplification reagents are in solid form (e.g., lyophilized, dried, crystallized, air jetted). In certain embodiments, one or more amplification reagents are in the form of an amplification pellet or tablet. The amplification pellet or tablet may comprise any amplification reagent described herein.

In some embodiments, the amplification pellet or tablet is shelf stable for a relatively long period of time. In certain embodiments, the amplification pellet or tablet is shelf stable for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the amplification pellet or tablet is shelf stable for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the amplification pellet or tablet is thermostabilized and is stable across a wide range of temperatures. In some embodiments, the amplification pellet or tablet is stable at a temperature of at least 0° C., at least 10° C., at least 20° C., at least 37° C., at least 40° C., at least 50° C., at least 60° C., at least 65° C., at least 70° C., at least 80° C., at least 90° C., or at least 100° C. In some embodiments, the amplification pellet or tablet is stable at a temperature in a range from 0° C. to 10° C., 0° C. to 20° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

In some embodiments, an isothermal amplification method described herein comprises applying heat to a sample. In certain instances, an amplification method comprises applying an amplification heating protocol comprising heating the sample at one or more temperatures for one or more time periods using any heater described herein. In some embodiments, an amplification heating protocol comprises heating the sample at a first temperature for a first time period. In certain instances, the first temperature is at least 30° C., at least 32° C., at least 37° C., at least 50° C., at least 60° C., at least 65° C., at least 70° C., at least 80° C., or at least 90° C. In certain instances, the first temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, or at least 30 minutes. In certain instances, the first time period is in a range from 1 to 3 minutes, 1 to 5 minutes, 1 to 10 minutes, 1 to 15 minutes, 1 to 20 minutes, 1 to 30 minutes, 3 to 5 minutes, 3 to 10 minutes, 3 to 15 minutes, 3 to 20 minutes, 3 to 30 minutes, 5 to 10 minutes, 5 to 15 minutes, 5 to 20 minutes, 5 to 30 minutes, 10 to 20 minutes, 10 to 30 minutes, or 20 to 30 minutes.

In some embodiments, an amplification heating protocol comprises heating the sample at a second temperature for a second time period. In certain instances, the second temperature is at least 30° C., at least 32° C., at least 37° C., at least 50° C., at least 60° C., at least 65° C., at least 70° C., at least 80° C., or at least 90° C. In certain instances, the second temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, an amplification heating protocol comprises heating the sample at a third temperature for a third time period. In certain instances, the third temperature is at least 30° C., at least 32° C., at least 37° C., at least 50° C., at least 60° C., at least 65° C., at least 70° C., at least 80° C., or at least 90° C. In certain instances, the third temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the third time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the third time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes.

In some embodiments, a lysis heating protocol may comprise heating a sample at one or more additional temperatures for one or more additional time periods.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence. In some embodiments, LAMP is combined with a reverse transcription reaction, and referred to as RT-LAMP.

In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample. Exemplary LAMP primers for detection of a SARS-CoV-2 nucleic acid sequence are provided in Table 1 below.

TABLE 1
Exemplary LAMP Primers (SARS-CoV-2)

| | Sequence | SEQ ID |
|---|---|---|
| Primer | (5′ to 3′) | NO: |
| F3_Set1 | CGGTGGACAAATTGTCAC | 1 |
| B3_Set1 | CTTCTCTGGATTTAACACACTT | 2 |
| Loop F_ | TTACAAGCTTAAAGAATGTCTG | 3 |
| Set1 | AACACT | |
| Loop B_ | TTGAATTTAGGTGAAACATTTG | 4 |
| Set1 | TCACG | |
| FIP1_Set1 | TCAGCACACAAAGCCAAAAATTTA | 5 |
| | TCTGTGCAAAGGAAATTAAGGAG | |
| BIP2_Set1 | TATTGGTGGAGCTAAACTTAAAGC | 6 |
| | CCTGTACAATCCCTTTGAGTG | |
| FIP2_Set1 | TCAGCACACAAAGCCAAAAATTTA | 7 |
| | TTTTTCTGTGCAAAGGAAATTAAG | |
| | GAG | |
| BIP2_Set1 | TATTGGTGGAGCTAAACTTAAAGCC | 8 |
| | TTTTCTGTACAATCCCTTTGAGTG | |
| F3_Set2 | TGCTTCAGTCAGCTGATG | 9 |
| B3_Set2 | TTAAATTGTCATCTTCGTCCTT | 10 |
| FIP_Set2 | TCAGTACTAGTGCCTGTGCCCACA | 11 |
| | ATCGTTTTTAAACGGGT | |
| BIP_Set2 | TCGTATACAGGGCTTTTGACATCT | 12 |
| | ATCTTGGAAGCGACAACAA | |
| Loop F_ | CTGCACTTACACCGCAA | 13 |
| Set2 | | |
| Loop B_ | GTAGCTGGTTTTGCTAAATTCC | 14 |
| Set2 | | |

In some embodiments, the LAMP reagents comprise a FIP and a BIP for one or more target nucleic acids. In some embodiments, the FIP and BIP each have a sequence that is at least 90%, at least 95%, at least 96%, at least 97%, at least 98%, at least 99%, at least 99.5%, or 100% identical to a primer sequence provided in Table 1. In some embodiments, the concentrations of FTP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FTP and BIP are each in a range from 0.5 aM to 1 μM, 0.5 μM to 1.5 μM, 0.5 μM to 2.0 μM, 1 μM to 1.5 μM, 1 μM to 2 μM, or 1.5 μM to 2 μM.

In some embodiments, the LAMP reagents comprise an F3 primer and a B3 primer for one or more target nucleic acids. In some embodiments, the F3 primer and the B3 primer each have a sequence that is at least 90%, at least 95%, at least 96%, at least 97%, at least 98%, at least 99%, at least 99.5%, or 100% identical to a primer sequence provided in Table 1. In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acids. In some embodiments, the forward loop primer and the backward loop primer each have a sequence that is at least 90%, at least 95%, at least 96%, at least 97%, at least 98%, at least 99%, at least 99.5%, or 100% identical to a primer sequence provided in Table 1. In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM.

In some embodiments, the LAMP reagents comprise LAMP primers designed to amplify a human or animal nucleic acid that is not associated with a pathogen, a cancer cell, or a contaminant (e.g., a control nucleic acid). In some such embodiments, the human or animal nucleic acid may act as a control. For example, successful amplification and detection of the control nucleic acid may indicate that a sample was properly collected, and the diagnostic test was properly run.

In some embodiments, the control nucleic acid is a nucleic acid sequence encoding human RNase P. Exemplary LAMP primers for RNase P are shown in Table 2. In some instances, the one or more LAMP reagents comprise at least four primers that each have a sequence that is at least 90%, at least 95%, at least 96%, at least 97%, at least 98%, at least 99%, at least 99.5%, or 100% identical to a primer sequence provided in Table 2.

TABLE 2
Exemplary RNase P Primers

| | | SEQ ID |
|---|---|---|
| Primer | Sequence (5′ to 3′) | NO: |
| F3 | TTGATGAGCTGGAGCCA | 15 |
| B3 | CACCCTCAATGCAGAGTC | 16 |
| FTP | GTGTGACCCTGAAGACTCGGTT | 17 |
| | TTAGCCACTGACTCGGATC | |
| BIP | CCTCCGTGATATGGCTCTTCGTT | 18 |
| | TTTTTCTTACATGGCTCTGGTC | |
| Loop F | HEX- | 19 |
| | ATGTGGATGGCTGAGTTGTT | |
| Loop B | CATGCTGAGTACTGGACCTC | 20 |
| Quencher | CAGCCATCCACAT-BHQ1 | 21 |

In some embodiments, one or more LAMP primers comprise a label. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay). In certain embodiments, the label is a fluorescent label. In some instances, the fluorescent label is associated with a quenching moiety that prevents the fluorescent label from signaling until the quenching moiety is removed. In certain embodiments, a LAMP primer is labeled with two or more labels.

The disclosure is based, in part, on LAMP amplification buffers comprising biotinylated oligonucleotides (e.g., biotinylated primers). In some embodiments, a LAMP amplification buffer comprises one or more target nucleic acid-specific biotinylated primers. In some embodiments, a LAMP amplification buffer comprises one or more control nucleic acid-specific biotinylated primers. The biotinylated primers bay be outer primers, inner primers and/or loop primers. In some embodiments, a target nucleic acid-specific biotinylated primer and a control nucleic acid-specific biotinylated primer are present in non-equimolar concentrations in a buffer solution. In some embodiments, the ratio of concentration of a target nucleic acid-specific biotinylated primer to a control nucleic acid-specific biotinylated primer ranges from about 1:5 to about 5:1. In some embodiments, the ratio of concentration of a target nucleic acid-specific biotinylated primer to a control nucleic acid-specific biotinylated primer is about 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, or 3.0:1. In some embodiments, the ratio of concentration of a target nucleic acid-specific biotinylated primer to a control nucleic acid-specific biotinylated primer ranges from about 1:5 to about 5:1. In some embodiments, the ratio of concentration of a target nucleic acid-specific biotinylated primer to a control nucleic acid-specific biotinylated primer is about 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, or 1:3.0. In some embodiments, an amplification buffer comprises a total concentration of biotinylated oligonucleotides that is less than 200 nM. In some embodiments, an amplification buffer comprises a total concentration of biotinylated oligonucleotides that ranges from about 1 nm to 10 nM, 2 nM to 25 nM, 5 nm to 30 nM, 10 nm to 50 nM, 20 nm to 75 nM, 50 nM to 150 nM, or 100 nM to 200 nM.

In some embodiments, the disclosure relates to the recognition that reducing (attenuating) the amount or concentration of one labeled primer in an amplification mixture relative to the other labeled primers in the mixture allows for the resulting amplicon mixture to be added to a lateral flow assay device directly, without the need for dilution. Thus, in some embodiments, an amplification mixture comprises a first labeled primer (e.g., a loop primer labeled with a first hapten, such as FITC), wherein the concentration of that primer ranges from about 5 nM to about 25 nM, In some embodiments, the concentration of the first primer in the amplification mixture is about 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, or 25 nM. In some embodiments, an amplification mixture comprises a primer comprising a second hapten (e.g., a digoxigenin (DIG) molecule), wherein the concentration of a second hapten in a buffer solution ranges from about 5 nM to about 100 nM. In some embodiments, the concentration of the first primer in the amplification mixture is about 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, or 100 nM.

In some embodiments, a buffer solution further comprises one or more sets (e.g., pairs) of outer primers. In some embodiments, at least one set (e.g. pair) of outer primers specifically binds to a target nucleic acid. In some embodiments, at least one set of outer primers specifically binds to a control nucleic acid.

In some embodiments, a buffer solution further comprises one or more additional sets (e.g., pairs) of internal primers. In some embodiments, at least one set (e.g., pair) of internal primers specifically binds to a target nucleic acid. In some embodiments, at least one set (e.g., pair) of internal primers specifically binds to a control nucleic acid.

In some embodiments, an amplification buffer comprises a DNA polymerase with high strand displacement activity. Non-limiting examples of suitable DNA polymerases include a DNA polymerase long fragment (LF) of a thermophilic-bacteria, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO₄ is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5 M, 1.0 M to 1.2 M, or 1.0 M to 1.5 M.

Aspects of the disclosure relate to methods of producing secondary reaction products during LAMP reactions that are useful surrogates for detection of LAMP amplicons during lateral flow assays (LFA). The disclosure is based, in part, on the use of oligonucleotide strand displacement (OSD) probes which allow for the direct application of reacted NAA mixes to lateral flow strips without any intervening dilution or sample preparation steps. In some embodiments, the methods comprise performing secondary nucleic acid reactions alongside the primary NAA reaction responsible for amplifying the diagnostic target sequence (e.g., an isothermal amplification reaction such as LAMP). In some embodiments, the secondary nucleic acid reactions result in the formation of controllable amounts of secondary products of desired size, structure and chemical labelling position/stoichiometry, optimized for performance as ideal LFA analytes according to these properties.

Accordingly, in some aspects, the disclosure provides a method for producing lateral flow assay (LFA) detection products, the method comprising performing an isothermal amplification reaction (e.g., using a method as described herein) in the presence of one or more mediator displacement probes (MDPs) under conditions under which a dual-labeled LFA detection product is produced.

In some embodiments, the mediator displacement probes comprise a first single stranded oligonucleotide Med probe comprising a detectable label, and a second hemi-duplex oligonucleotide probe comprising a single stranded target sequence-specific portion contiguously linked (e.g., sharing the same phosphate-based backbone) with a MedC oligonucleotide that is complementary to the Med probe. In some embodiments, the Med probe is annealed (e.g., hybridized) to the MedC portion of the second hemi-duplex oligonucleotide probe. In some embodiments, the Med probe further comprises an extension blocking group on its 3′ end. In some embodiments, the mediator probe set further comprises a Universal Reporter probe comprising an oligonucleotide sequence that is complementary to the Med probe. In some embodiments, the Universal Reporter probe comprises a detectable label. In some embodiments, the detectable label is positioned on the 3′ end of the Universal Reporter. In some embodiments, the Universal Reporter probe further comprises an extension blocking group on its 3′ end. In some embodiments, the target sequence-specific portion of the second hemi-duplex oligonucleotide probe comprises a region of complementarity with a human gene. In some embodiments, the human gene is a RNAse P gene. In some embodiments, the target sequence-specific portion of the second hemi-duplex oligonucleotide probe comprises a region of complementarity with a pathogen. In some embodiments, the pathogen is SARS-CoV-2.

The ratio of mediator displacement probe to Universal Reporter in an amplification reaction may vary. In some embodiments, the concentration (e.g., as expressed in nM) of a mediator displacement probe to a Universal Reporter in an amplification mixture ranges from about 1:3 to about 3:1. In some embodiments, the concentration (e.g., as expressed in nM) of a mediator displacement probe to a Universal Reporter in an amplification mixture is about 1:3, 1:2, 1:1, 2:1, or 3:1. In some embodiments the concentration of a mediator displacement probe in an amplification mixture ranges from about 0.4 nM to about 500 nM 0.4 nM to about 500 nM (e.g., about 0.4 nM, 1 nM, 10 nM, 50 nM, 100 nM, 200 nM, 300 nM, 400 nM, or 500 nM). In some embodiments, the concentration of a Universal Reporter in an amplification mixture ranges from about 0.4 nM to about 500 nM (e.g., about 0.4 nM, 1 nM, 10 nM, 50 nM, 100 nM, 200 nM, 300 nM, 400 nM, or 500 nM).

A “dual-labeled LFA detection product”, as used herein, refers to a double stranded oligonucleotide comprising a single stranded Med probe hybridized to a single stranded Universal Reporter, wherein each of the Med probe and the Universal Reporter comprise a label that is detectable by a lateral flow assay (LFA). In some embodiments, each label of a dual-labeled LFA detection product is selected from biotin, FAM, and DIG. In some embodiments, the strand of the dual-labeled LFA detection product corresponding to the Med probe further comprises an extension blocking group. Without wishing to be bound by any particular theory, inclusion of an extension blocking group on the 3′ end of a dual-labeled LFA detection product reduces the amount of background signal present during subsequent detection of the labels during LFA.

Aspects of the disclosure relate to the recognition that use of mediator displacement probes during LAMP to produce dual-labeled LFA detection products allows for improved multiplexing of LAMP reactions. Thus, in some embodiments, a LAMP reaction comprises 2, 3, 4, 5, 6, 7, 8, 9, 10, or more different LAMP primer sets, and 2, 3, 4, 5, 6, 7, 8, 9, 10, or more different mediator displacement probe sets. In some embodiments, each of the different mediator displacement probe sets comprises a target sequence-specific sequence portion that corresponds with the target sequence-specific portions of the LAMP primer sets. In some embodiments, the sequences of the target sequence-specific sequence portions of the mediator displacement probe sets are at least 80%, 90%, 95%, 99%, or 100% identical to the forward Loop primers or reverse Loop primers of the corresponding LAMP primer sets.

### Detection

In some embodiments, amplified nucleic acids (i.e., amplicons of target nucleic acids or control nucleic acids) may be detected using any suitable methods. In some embodiments, one or more target nucleic acid sequences are detected using a lateral flow assay strip. In some embodiments, one or more target nucleic acid sequences are detected using a colorimetric assay.

Aspects of the disclosure relate to methods that result in detection of one or more signals on a lateral flow assay that are brighter (e.g., more intense, more easily visible to an unaided eye, etc.) than lateral flow assay signals produced using previous methods. In some embodiments, signals (e.g., colored bands) produced using isothermal amplification methods described herein are between 10% and 100% (10, 20, 30, 40, 50, 60, 70 80, 90, 95, 99, or 100%) brighter than lateral flow signals produced using previously available isothermal amplification methods.

In some embodiments, one or more target nucleic acid sequences are detected using a lateral flow assay strip (e.g., in a “chimney” detection component, a cartridge, a blister pack). In some embodiments, a fluidic sample (e.g., fluidic contents of a reaction tube, a reagent reservoir, and/or a blister pack chamber) is transported through the lateral flow assay strip via capillary action. In some embodiments, the fluidic sample may comprise labeled amplicons.

In some embodiments, the fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

In some embodiments, the fluidic sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip. Any excess fluid may flow through the fourth sub-region.

In some embodiments, the disclosure relates to rapid, self-administrable tests for detecting the presence of one or more target nucleic acids derived from one or more pathogens. A “self-administrable” test refers to a test in which all testing steps are performed by the subject of the test. In some embodiments, a self-administrable test is performed by a subject at a location that is not a medical facility (e.g., a hospital, physicians' office, nurse's office, etc.). In some embodiments, a self-administrable test is performed at the subject's home. In some embodiments, a “rapid test” refers to a test in which all testing steps (e.g., sample collection, lysis, isothermal amplification, detection, etc.) may be completed in less than 3 hours, less than 2 hours, or less than 1 hour. In some embodiments, a rapid test is completed (e.g., one of more nucleic acids are detected) in less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 minutes.

As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line. In some embodiments, one or more target nucleic acid sequences are detected using a colorimetric assay. In certain embodiments, for example, a fluidic sample is exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid is not present, and the sample is negative. In some embodiments, the colorimetric assay may be a colorimetric LAMP assay; that is, the LAMP reagents may react in the presence or absence of a target nucleic acid sequence (e.g., from SARS-CoV-2) to turn one of two colors.

### Examples

### Example 1

Lateral flow immunoassays are simple to use diagnostic assays that can quickly and visibly report the presence of absence of a target analyte (e.g. a virus or pathogen). Thus, they are attractive and commonly used components of affordable point-of-care diagnostic tests. Nucleic acid lateral flow immunoassays (NALFIA) are used specifically to detect nucleic acid amplicons that carry one or more chemical labels. Prior to performing NALFIA, amplicons are commonly diluted in a lateral flow “running buffer.” Alternatively, a small volume of amplicons is loaded on the lateral flow strip and subsequently “chased” with a larger volume of lateral flow running buffer. Although, pre-mixing or chasing sample with running buffer results in optimal lateral flow performance, this “dilution” step adds an extra step to the diagnostic workflow and requires one or more fluid transfer steps, often involving small volumes.

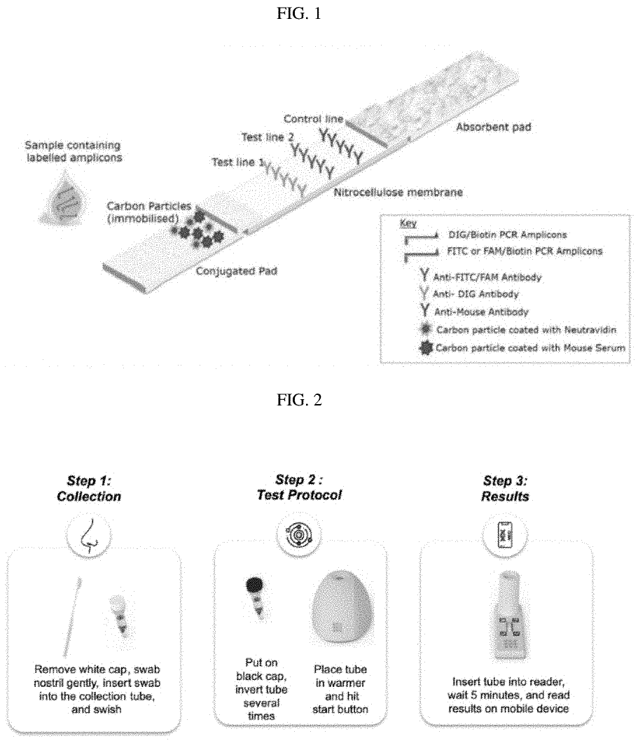

Reverse Transcription Loop-mediated isothermal amplification (RT-LAMP) is a single-tube, low-cost technique for the amplification of DNA and/or RNA. RT-LAMP can be performed using modified DNA oligonucleotide primers such that the resulting amplicon carries one or more chemical labels. For example, RT-LAMP may be performed using a mixture of unmodified DNA oligonucleotide primers and primers with FITC and/or biotin labels. In this manner, RT-LAMP can produce dual-labeled (e.g. FITC and BIOT) amplicons that can readily be detected by NALFIA, where streptavidin detection particle conjugates bind the biotin label, and labeled products form a bright line at an anti-FITC antibody line on the test strip. A multiplexed LAMP reaction with alternatively labeled primer sets may generate distinctly labeled amplicons and thus light up distinct lines on a lateral flow test strip from a single-tube reaction. For example, labeling one amplicon with digoxigenin (DIG) and biotin and a second amplicon with FITC and biotin can generate multiple signals on a single lateral flow strip using a single streptavidin conjugate particle (FIG. 1). However, RT-LAMP generated amplicons are commonly diluted in lateral flow running buffer prior to NALFIA.

In some aspects, the disclosure relates to a simple, single-tube SARS-CoV-2 test where patient material is directly introduced into an amplification reaction and that amplification reaction produces amplicons only in the presence of SARS-CoV-2 nucleic acid, as indicated by a visual NALFIA result (FIG. 2). The test may be a multiplexed RT-LAMP reaction that simultaneously detects the presence of a control nucleic acid (e.g., a human gene) and SARS-CoV-2 nucleic acids in the sample, which enables the inclusion of a positive control on every test. Despite the potential for a single-tube, simple RT-LAMP based SARS-CoV-2 assay, in order to generate strongly visible bands, amplicons are typically diluted before their application to a lateral flow device. Direct addition of amplicons generated by RT-LAMP to a lateral flow strip without any intervening dilution steps would therefore represent a considerable improvement to the ease of use of a diagnostic kit for SARS-CoV-2.

This example describes direct addition of amplicons to a lateral flow strip without any intervening dilution steps, which represents a considerable improvement to the ease of use of NALFIA diagnostics, for example diagnostics for detecting whether or not a subject is infected with SARS-CoV-2.

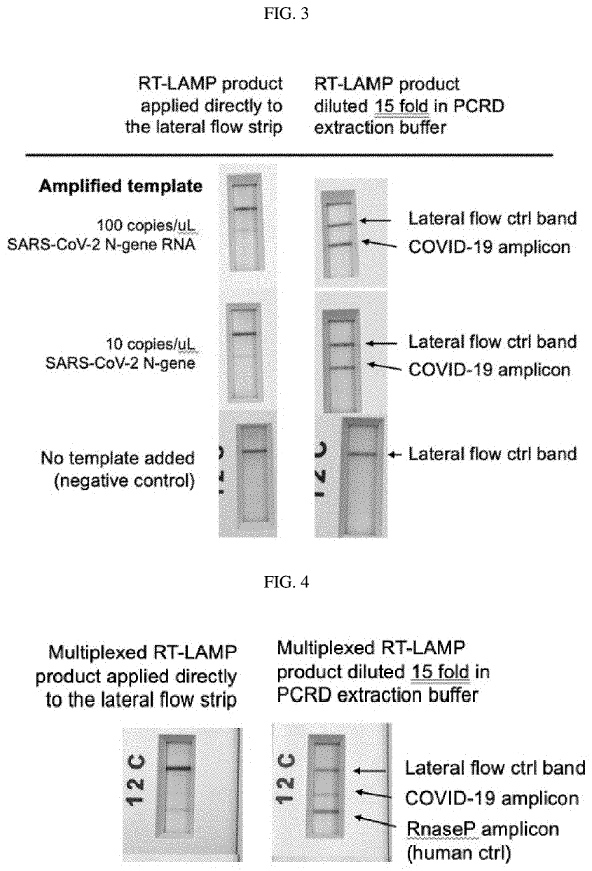

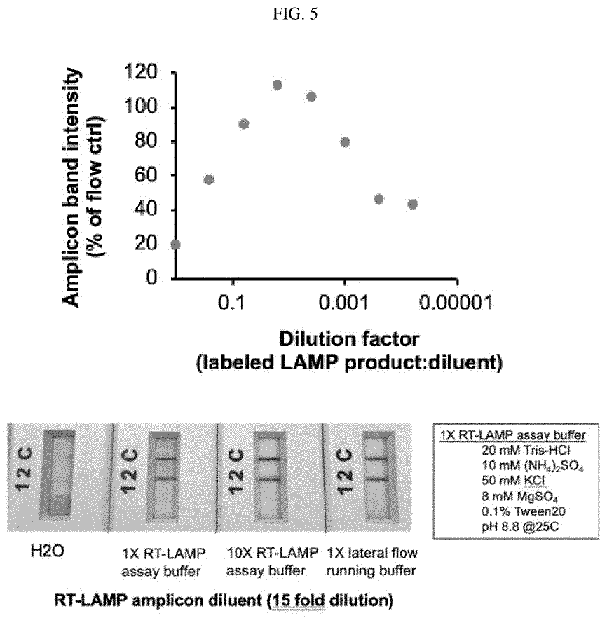

The disclosure is based, in part, on the observation that RT-LAMP products that are directly applied to lateral flow strips result in the development of visible, colored bands as expected. Although the lateral flow control band that detects fluid flow (not nucleic acid amplicons) develop at high intensity, amplicon-specific bands develop at an intensity that is far lower than is typical for lateral flow applications. Dilution of the RT-LAMP amplicons in a diluent solution prior to their application on a lateral flow strip results in a much-improved lateral flow result, with higher contrast bands that approach the intensity of the lateral flow control (FIG. 3). Dilution also results in a brighter intensity for visible bands generated from multiplexed labeled nucleic acid amplicons, where bands are so dim in the absence of dilution that the test result may be interpreted incorrectly—e.g., a negative COVID result in the absence of dilution prior to NALFIA and a positive COVID result when dilution is performed (FIG. 4).

To generate bright, visible bands on NALFIA, RT-LAMP products can tolerate a wide range of dilution ratios (FIG. 5). Initially, increased dilution results in ever increasing lateral flow band intensity. At high dilution ratios, the amplicon is so dilute that band intensity signal begins to decrease. Therefore, there is an optimal dilution factor that reduces the concentration of RT-LAMP product in diluent to the degree that generates bright visible lateral flow bands without diluting so much that the RT-LAMP product cannot readily be detected.

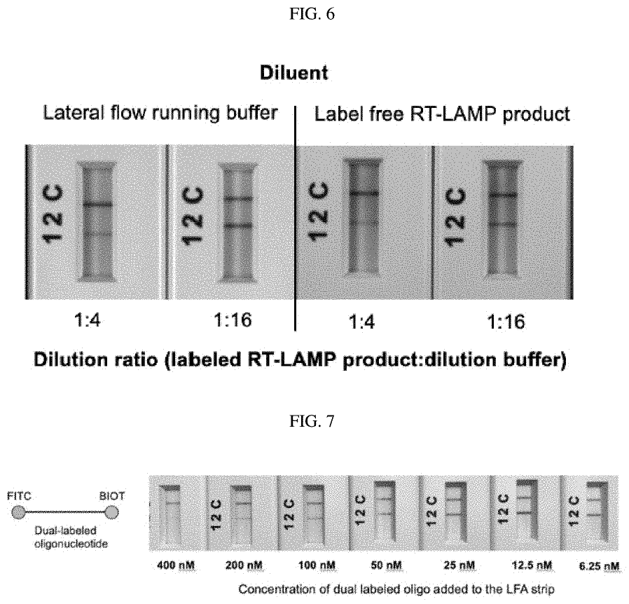

Although dilution of RT-LAMP amplicons is important for the evolution of bright lateral flow bands, it was identified that the diluent solution is flexible. Alternative diluents to the lateral flow running buffers supplied by flow strip manufacturers can work equivalently well at increasing lateral flow visible band signal. Although water is not a functional diluent due to the absence of a surfactant, RT-LAMP assay buffer, which includes the surfactant Tween20 at 0.1%, whether at 1× strength or highly concentrated, may serve as an amplicon diluent (FIG. 6). This means that a single buffer can support not only multiplexed RT-LAMP amplification but also visible lateral flow readout. Furthermore, it was determined that the LAMP reaction itself can serve as a diluent. An RT-LAMP reaction that generates labeled amplicons detectable by NALFIA (e.g. FITC- and BIOT-labeled) may be diluted into an RT-LAMP reaction run with all unmodified oligonucleotide primers (no detectable amplicons generated). This dilution can generate an equivalent increase in visible band signal as dilution into a lateral flow running buffer supplied by a strip manufacturer (FIG. 7). This is an important finding that indicates that dim bands observed from direct RT-LAMP application to lateral flow strips does not result from a lack of lateral flow running buffer elements in solution or from byproducts of RT-LAMP, but rather the dim bands are specific to the inclusion of labeled nucleic acids in solution. In other words, the experimental data strongly indicate that dim bands result from an overabundance of labeled amplicons, and darker lateral flow bands are realized by decreasing the concentration of those labeled amplicons via dilution into an unlabeled solution.

Collectively, these findings described above point to a counterintuitive solution to the “dim band problem”—namely that in the absence of dilution post-amplification, reduction in the concentration of labeled nucleic acid could actually result in an increase in visible band signal on lateral flow. In other words, by adding less labeled material, it is possible to produce more contrast in a given lateral flow band pattern. Reduction in labeled material resulting in brighter lateral flow signal was confirmed in two ways.

First, an oligonucleotide bearing two labels, a hapten and a biotin, was produced such that the dual-labeled oligo itself was sufficient for generating a signal on an NALFIA. Surprisingly, it was observed that the dual-labeled oligo gave maximum signal intensity on lateral flow at 6.25 nM. By loading more dual labeled oligo (fixed volume, but increasing concentration) lateral flow bands of decreasing intensity were generated (FIG. 7). This indicates that excess dual-labeled amplicon can overwhelm the absolute quantity of streptavidin conjugate and anti-hapten antibody in the lateral strip and hurt the evolved visible signal.

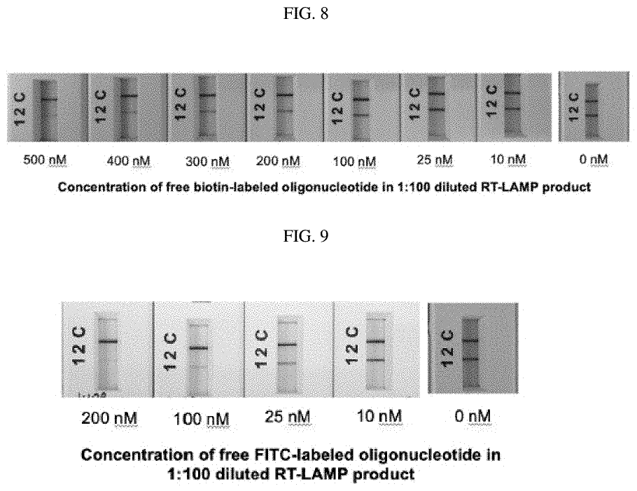

In addition to work with the dual-labeled oligo, an RT-LAMP amplicon was diluted to generate bright bands, and then single-label primers were added back into solution to investigate whether the signal would dim. Addition of either conjugate detection primers (e.g. biotin labeled) (FIG. 8) or hapten-labeled primers (e.g. FITC labeled) (FIG. 9) resulted in dimmer signals. These data indicate that if RT-LAMP reactions contain either unspent single-label primers or generate amplicons with single and not dual labels, that these single-label products can collectively result in dimmed lateral flow signal.

RT-LAMP with Attenuated Loops

RT-LAMP involves the amplification of a DNA or RNA target using 2 inner oligonucleotide primers, termed “FIP” and “BIP”, two outer oligonucleotide primers, termed “F3” and “B3”, and two optional accelerant primers, termed “LoopF” and “LoopB.” It is possible to generate a dual labeled amplicon by RT-LAMP for detection on NALFIA by chemically labeling pairs of these primers. “LoopF” and “LoopB” are typically the most common pair to carry labels, although “FIP” and “BIP” and combinations of “FIP” and “Loop” primers may be labeled instead.

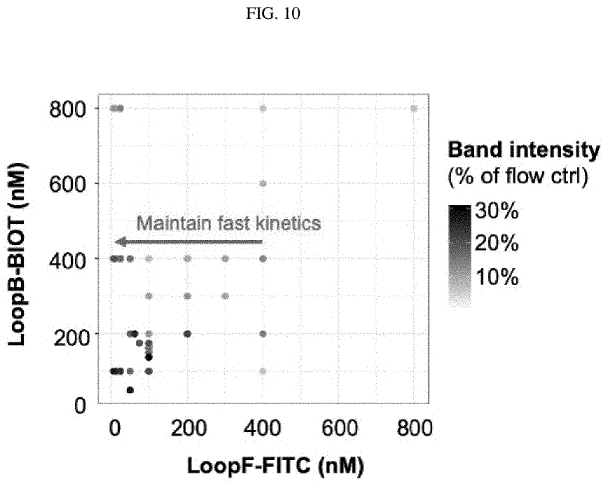

It was observed that reactions involving attenuated (e.g., reduced) concentrations of “LoopF” and “LoopB” may be applied directly to an NALFIA and generate brighter, more visible and human-readable bands. The lower the concentrations of labeled loop primers, the brighter the resulting bands when the amplicons are detected by lateral flow (FIG. 10).

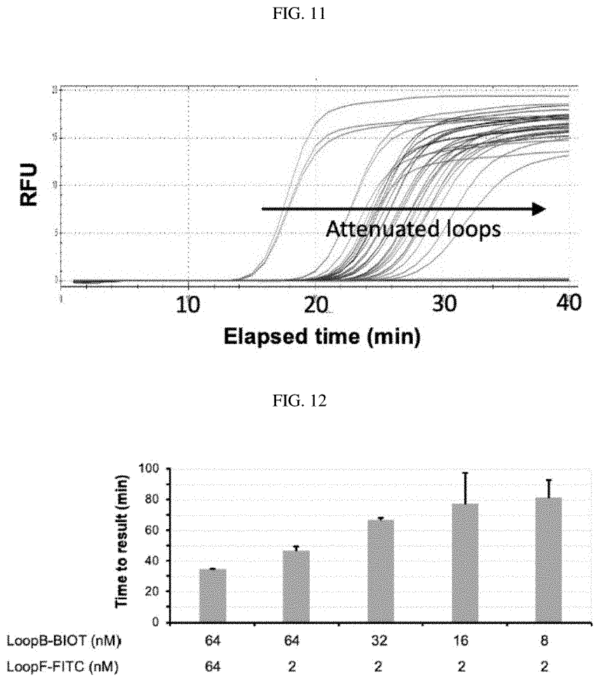

Despite their benefit in enhancing visible development of lateral flow strips, it was observed that attenuated “LoopF” and “LoopB” concentrations result in a slower amplification reaction (FIG. 11). Loss in the amplification speed was minimized by tuning the concentration of loops such that one loop is preserved at elevated concentration relative to the other rather than lowering both loops in an equimolar fashion. Reduction of a single loop by many fold results in less of a slowdown in amplification than simultaneous reduction in both loops. As shown in FIG. 11, reduction of LoopF-FITC from 64 nM to 2 nM (32× drop) with maintenance of LoopB-BIOT at 64 nM increases the assay time from 35 to 47 min (34%). In contrast, lowering LoopF-FITC to 2 nM (32×) and lowering LoopB-BIOT to even just 8 nM (8×) increases the assay time from 35 to 81 min (131%) (FIG. 12). Therefore, there is a distinct benefit of retaining at least one loop at as elevated a concentration as possible to maintain fast amplification kinetics.

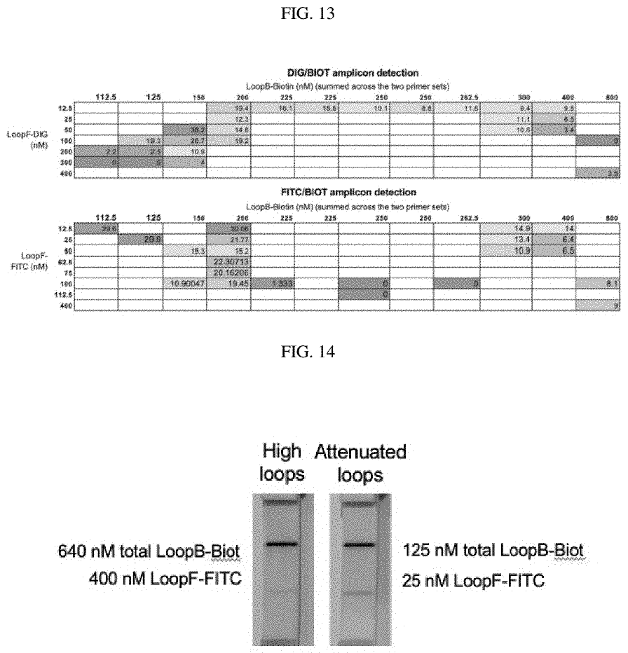

Using labeled primers and dual-labeled oligos, the lateral flow signal landscape on commercially available lateral flow strips was mapped. It was observed that to maximize signal evolution by lateral flow to detect SARS-CoV-2, loop concentrations are tuned such that the total concentration of biotinylated oligonucleotides in the reaction is <200 nM, FITC labeled primer to be <25 nM and DIG labeled primer to be <100 nM (FIG. 13).

One possible recipe that elicits bright bands in the absence of dilution is to have LoopB-BIOT at 100 nM and LoopF-hapten-1 at 25 nM for one primer set and a second primer set to have LoopB-BIOT at 25 nM and LoopF-hapten-2 at 100 nM, where hapten-1 and hapten-2 are distinct (such as FITC and DIG). Two primer sets, one that detects the human RNase P (RP) gene with DIG labeled LoopF (LF) and BIOT labeled LoopB (LB) and a second primer set that detects SARS-CoV-2 (CV-19) with FITC labeled LoopF (LF) and BIOT labeled LoopB (LB) were prepared. The concentrations of these loops were set such that RP-LF-DIG was at 100 nM, RP-LB-BIOT was at 25 nM, CV19-LF-FITC was at 25 nM and CV19-LB-BIOT was at 100 nM. These loop concentrations satisfy the conditions described in FIG. 13 for bright bands. This attenuated loop buffer recipe results in a significant brightening of the lateral flow strip as compared to a recipe with elevated labeled loop concentrations (FIG. 14).

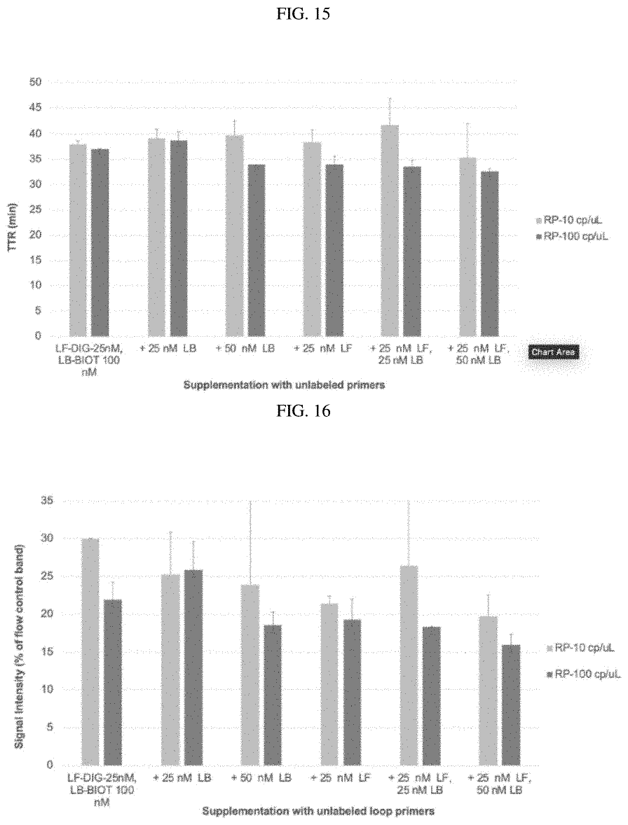

Given the kinetic slow-down that results from the reduction of labeled primers in the attenuated loop mix, labeled loop reaction mixtures were supplemented with unlabeled loop primers in the hopes of accelerating the reaction without diminishing the band intensity. It was observed that spiking unlabeled loops into an attenuated loop RT-LAMP reaction can accelerate the amplification (FIG. 15) but also reduces the lateral flow band intensity (FIG. 16).

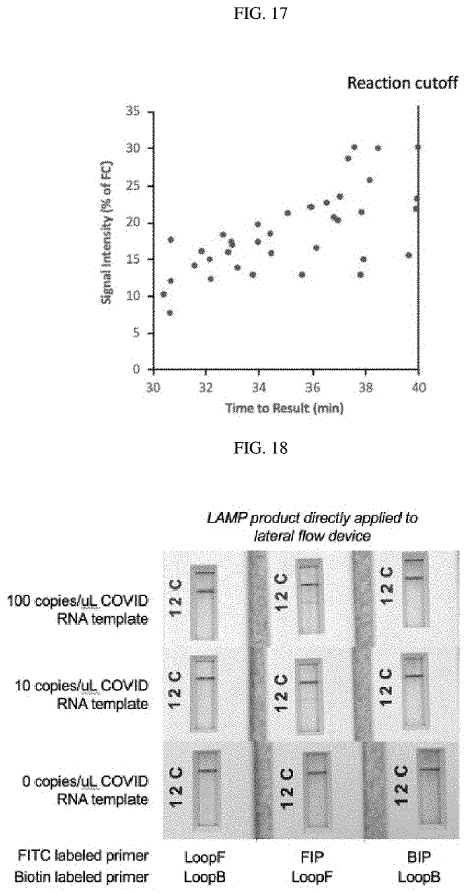

Whether there is a disadvantage to increased speed from a lateral flow band brightness perspective was investigated. Surprisingly, it was observed that the closer a reaction enters the exponential amplification phase to the conclusion of the assay and application to the lateral flow strip, the brighter the evolved band intensity signal. This indicates that limiting the total amplified product is critical for achieving bright lateral flow bands in the absence of dilution (FIG. 17).

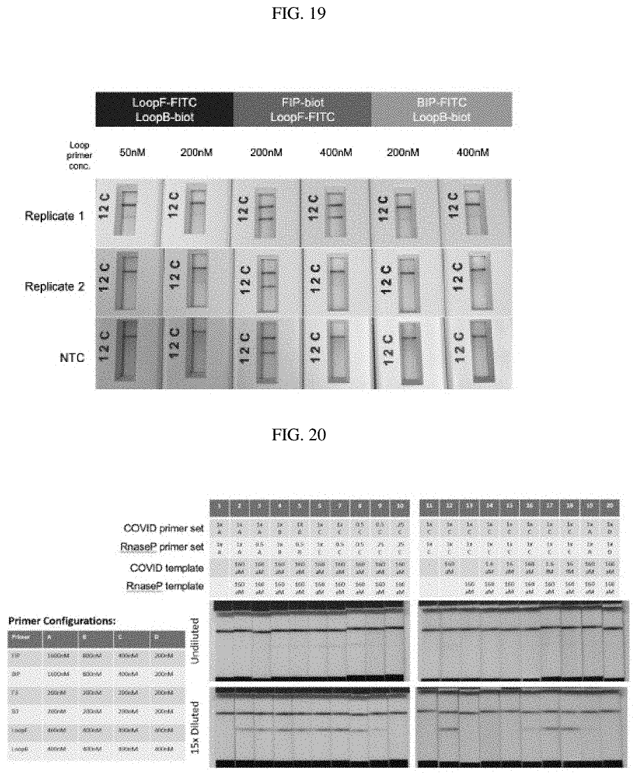

Whether labels could be added to other primers aside from loop primers to generate signal on lateral flow and if those alternative labeling schemes could be used to modulate band brightness was investigated. Three different pairs of LAMP primers: (i) LoopF and LoopB (ii) FIP and LoopF and (iii) BIP and LoopB, were labeled. For certain primer sets, it was observed that FIP/LoopF labeled amplification products resulted in superior brightness when directly applied to lateral flow strips (FIG. 18). For other primer sets, it was observed that the LoopF/FIP labeling led to the development of false positive signals even in the absence of template. (FIG. 19). Alternative labeling schemes may be used to modulate lateral flow signal intensity, but that trends are primer-set dependent.

Beyond changing the concentrations and identities of the labeled primers in the reaction, altering the concentrations of the unlabeled primers was also investigated. It was observed that lowering the concentration of FIP and BIP primers can improve band brightness for “undiluted” samples directly applied to the flow strip with no intervening steps (FIG. 20). Since band brightness is inversely correlated with the mass of labeled DNA produced by RT-LAMP amplification reactions, lowering FIP/BIP concentrations may reduce DNA yield, thereby increasing band brightness.

### Example 2

Labeled Strand Displacement Probes that Logically Process and Report the Diagnostic Result on Lateral Flow

Oligonucleotide strand-displacement (OSD) probes were also investigated as a solution to the “dim band problem” described in Example 1. Oligonucleotide stand-displacement probes are generally known, for example as described by Jiang et al., Angew Chem Int Ed Engl. 2014 Feb. 10; 53(7): 1845-1848; Phillips et al., Anal Chem. 2018 Jun. 5; 90(11): 6580-6586; and Bhadra et al. (2020) doi.org/10.1101/2020.04.13.039941.

OSD strand-displacement probes, in some embodiments, consist of a labeled ‘short’ strand and an unlabeled ‘long’ strand. The short and long strands are annealed to form a hemi-duplex with a mostly double stranded region and a short, exposed single-stranded ‘toehold’. For the labeled short strand to be released to generate an LFA-compatible analyte, the short toehold region must bind to a complementary target sequence with sufficiently high affinity to drive the non-enzymatic strand-exchange reaction. Especially at the elevated temperatures (60-70° C.) at which LAMP is conventionally run, this stringent sequence recognition event translates into a very high specificity reaction/detection. Well-designed OSD probes are unlikely to undergo strand-exchange unless their exact target nucleic acid target sequence is present in the reaction mixture, providing a highly specific molecular “validation” of the identity of amplified nucleic acid products. This feature results in a very specific test and translates directly into a clinical benefit for reactions run as part of diagnostic kits.

For example since, OSD strand-displacement requires a nearly exact match of the toehold domain to its target sequence to trigger a robust strand-exchange cascade reaction resulting in generation of its corresponding lateral flow-detectable analyte. Therefore, it is a natural consequence of this design that method described herein may be used to distinguish between two very closely related target variants that differ at as few as a single position, especially if the polymorphism is positioned at the center of the toehold. Variant detection has a number of applications, including the differentiation of pathogenic microbial variants or identification of a high-risk, disease-causing somatic mutation. For example, certain strains of SARS-CoV-2 have emerged as variants of concern, including B.1.1.7, B.1.351 and P.1.

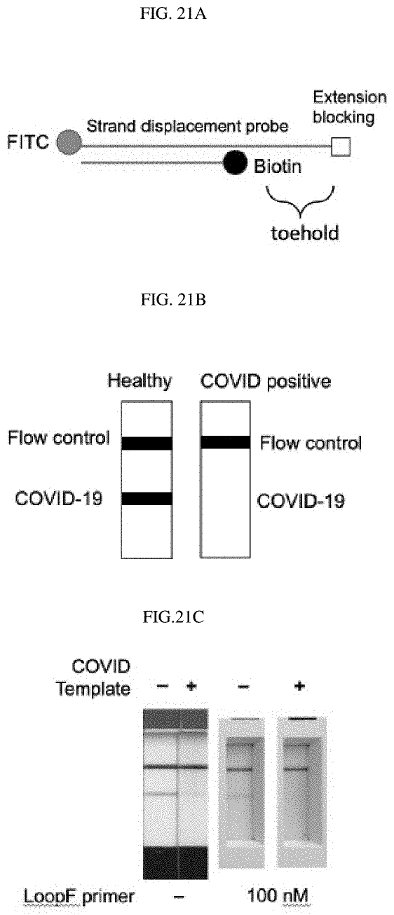

OSD probes were modified by labeling the duplex with a 5′ FITC on the top strand and a 5′ BIOT group on the bottom strand. Initially, the dual labeled duplex is intact, and strong bands can be seen on the LFA strip. The concentration of the OSD probe can be set to dial in a bright signal. As the duplex probe undergoes strand displacement, the two labels are separated. This results in a disappearance of the dual-labeled product and the disappearance of the lateral flow bands (FIGS. 21A-21C).

Dual-label OSD probes can be used in the presence of high loop primer concentrations. Thus, dual-labeled OSD probes could be used to achieve band bands on lateral flow for an undiluted reaction and maintain high LAMP performance. OSD probe data indicate that it is possible to apply a LAMP product directly to lateral flow and achieve a strong visible band signal without any dilution step.

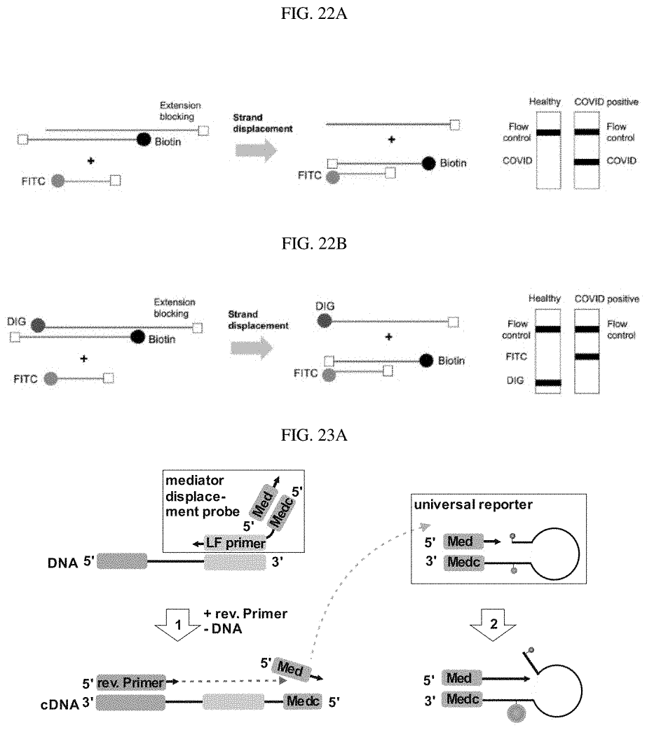

While OSD probes are promising solutions to the undiluted band problem, the approach described above is inverted—in other words, the presence of a band indicates no amplification and the absence of a band indicates amplification. Thus, strand displacement-initiated cascades may be used to invert OSD reporters such that strand displacement results in the formation of a dual-labeled oligo duplex rather than generates single-labels. For example, strand-displacement may release a labeled “bottom strand oligo” such that it would be free to anneal to a second single-stranded reporter oligo and generate a dual-labeled duplex (FIGS. 22A-22B). The dual-labeled duplex must only form in the presence of amplicon and not in a negative control reaction missing template. Therefore, the thermodynamics governing strand displacement should guarantee that it would only be favorable for the labeled duplex to form under conditions where OSD probe strand displacement has already occurred in the presence of amplicons.

### Example 3: Modified Mediator Displacement Probes

This example describes compositions and methods for the direct application of reacted NAA mixes to lateral flow strips without any intervening dilution or sample preparation steps. The disclosed methods work by incorporating secondary nucleic acid reactions alongside the primary NAA reaction responsible for amplifying the diagnostic target sequence. These secondary nucleic acid reactions result in the formation of controllable amounts of secondary products of desired size, structure and chemical labelling position/stoichiometry, optimized for performance as ideal LFA analytes according to these properties. In some embodiments, the methods enable direct readout of the sensitive and specific detection of a target nucleic acid by convenient one-step sandwich assay devices like LFA strips and permits multiplexed detection, enabling the parallel detection/readout of one or more target sequences.

Nucleic acid amplification tests (NAATs) enable fast, sensitive and specific detection of target nucleic acid sequences, including pathogenic viruses, bacteria and other organisms. Conventional NAAT tests are performed in high-complexity laboratories by trained technicians. However, decentralized and rapid point-of-care (POC) and non-laboratory NAAT testing is increasingly sought after. POC and non-lab NAAT tests are transformative since they permit diagnostic testing when and where it is needed and, for certain tests, can be performed by untrained operators. To maximize successful POC and non-lab NAAT test use, it is essential to minimize the number of total hands-on steps in the diagnostic workflow, minimize the opportunity for operator-introduced error and to maximize the likelihood of correct interpretation of the test results. It is also essential to prevent release of NAAT-amplified products, since aerosolized or dispersed nucleic acid amplicons may contaminate future tests and compromise their diagnostic accuracy.

Nucleic acid lateral flow immunoassays (NALFIA) are well-suited to POC and non-lab tests given that they are inexpensive and robust, and the test result is a simple-to-interpret visible band on a paper strip. Current state-of-the-art NALFIA tests require the use of a running buffer to ensure that the formed bands are bright enough to be readily seen. However, the addition of a buffer mixing step complicates the operating workflow and introduces an opportunity for operator error (insufficient, excessive or otherwise improper buffer addition; spillage). Buffer mixing also complicates efforts to prevent the distribution of amplified products in the environment as nucleic acids are readily aerosolized if they are exposed to the atmosphere during any mixing steps.

An incubated NAA reaction mix that can be directly applied to an LFA device with no requirement for separate running buffer addition would therefore represent a considerable improvement to the ease of use of diagnostic ‘kits’ that include a NAA step followed by detection/readout on an LFA strip. Methods described herein, in some embodiments, solve usability problems for diagnostic test kits both in terms of test result interpretation and test operation and solves accuracy challenges by facilitating the complete enclosure of the test. Importantly, this approach is universal—the NAA methods described in this disclosure may be applied to all one step immunoassay tests. The methods here are thus an improvement over alternative buffer-elimination strategies that would require changing the architecture and design of the LFA test itself.

Nucleic acid circuitry for transducing an amplification reaction into an alternative signal type is broadly useful. The methods described herein may be applied, for example, to the integration of multiple amplification inputs into a single output signal according to a predetermined logic—such as, a secondary product may only form if and only if two distinct molecular targets of amplification are present. Alternatively, secondary products of nucleic acid amplification reactions may be useful for detection via non-LFA based detection modes, including by fluorescence and electrochemistry.

In some embodiments, methods and compositions described herein mitigate the hook-effect (e.g., as described in Example 1) by (i) driving a secondary reaction alongside a primary nucleic acid amplification reaction such that the primary reaction generates, with high sensitivity and speed, an abundance of target nucleic acid amplification product not competent for binding to the specific binding/detection reagents of the LFA; and (ii) the secondary reaction generates a lower, controlled concentration of a different product competent for LFA binding/detection, appropriate to the dynamic range of the lateral flow device. This advantage eliminates the requirement for post-amplification mixing of the nucleic acid amplification product with a diluent buffer and thus vastly simplifies lateral flow test result generation and interpretation.

As described previously, traditional OSD probes undergo strand exchange reactions when their short, exposed single-stranded toehold regions exactly or nearly exactly match their target sequences. Thus, each and every traditional OSD probe must be designed with its specific target sequence in mind; a traditional OSD probe that undergoes strand displacement upon binding one molecular target cannot simply be reused and expected to undergo sequence-specific strand displacement upon interacting with a distinct molecular target. In fact, as noted, this remarkable specificity is considered a feature. This feature does however pose a challenge as new OSD probe sequences need to be designed for every unique molecular target. The requirement for custom design adds cost, time and uncertainty to the development of new molecular diagnostic assays.

In contrast, the modified mediator displacement probes described herein employ the use of ‘universal’ primer ‘extensions.’ These modular duplex primer extensions may be appended onto the 5′ end of arbitrary single stranded LAMP primers to convert them into hemi-duplex primers that undergo strand displacement any time the single stranded portion of the primer is involved in isothermal amplification. Displaced strands originating from the hemi-duplex primers in turn drive secondary strand displacement and/or hybridization reactions with additional probes, also universal in design, to realize the formation of ideal LFA analytes. In this manner, with a single ‘universal’ design for a duplex primer extension sequence, one may adapt arbitrary isothermal amplification primer sets to realize the direct application of reacted NAA mixes to lateral flow strips without any intervening dilution or sample preparation steps.

It has been observed that adding target analytes (labeled nucleic acid amplicons) that are too concentrated and outside of the acceptable range for the lateral flow assay causes the ‘hook effect’ to be realized due to competition for binding to detection conjugate and capture antibodies. The hook effect ultimately results in the formation of difficult to interpret “dim bands.” Since RT-LAMP and LAMP are highly productive amplification reactions that can produce 10\hat{ }10 copies of the target nucleic acid, direct application of NAA to LFA results in the realization of the ‘hook effect.’ Since the hook effect is driven by competition for sandwich formation between the target analyte, the detection conjugate and the capture probe, analytes that lack affinity to both the detection conjugate and the capture probe will not exhibit hook effect phenomena. Therefore, the application of off-target molecules to LFA is not similarly restricted. Any restrictions on the concentration of off-target analytes only stem from alternative effects background material might have on the LFA, e.g., by affecting flow through the device among other mechanisms. For example, one may apply concentrated, yet unlabeled ‘background’ nucleic acid to a nucleic acid lateral flow immunoassay (NALFIA) alongside the target analyte with no consequence on the target analyte detection over a very large range of concentrations of the background nucleic acid material.

The observation that unlabeled nucleic acids have a favorable concentration range over which they may be added as background material to an NALFIA while target analyte concentrations must be narrowly restricted is what directly the inspired innovation disclosed here: a method for detecting target LFA analytes that originate as side products from isothermal amplification reactions. A primary isothermal amplification reaction generates uncontrolled amounts of reacted NAA mix (with no affinity for the LFA detection and capture moieties), which in turn activates a secondary mechanism whereby a detectable LFA analyte is formed. By tuning the precise concentration of the components required for secondary product generation, the concentration of the resulting target analyte is maintained within the narrow range acceptable for LFA. As noted, the primary reaction may proceed uncontrolled as, even at saturation, the LFA is agnostic to the unlabeled reacted NAA mixes produced.

The disclosed methods for generating secondary products of amplification involve nucleic acid circuitry. Isothermal amplification triggers the separation and/or combination of individual nucleic acids strands in a programmatic manner according to pre-designed logic. In one embodiment, the primary amplification reaction catalyzes the release of a single stranded labeled nucleic acid. A primary amplification reaction may catalyze the release of an oligonucleotide strand in at least two ways. First, hemi-duplex primers consisting of a single stranded region with homology to the amplification target molecule and a 5′ duplex extension may be used. After the single stranded region primes extension by the polymerase and is effectively incorporated into the amplicon, the polymerase releases the annealed short strand from a hemi-duplex on a subsequent round of amplification and extension by strand-displacement. Alternatively, one may employ hemi-duplex probes that do not actively participate in the primary amplification process. The thermodynamically favorable hybridization of the hemi-duplex probe with its exposed single stranded toehold to the product of the primary amplification product may drive a strand exchange, reaction and subsequent release of the bound short strand.

By virtue of chemically labeling the nucleic acid strands involved in the circuitry described above and controlling the concentration of these molecules in the overall amplification reaction, it is possible to realize the formation of an appropriately concentrated dual-labeled nucleic acid product post-amplification that is detectable by a one-step sandwich immunoassay like LFA.

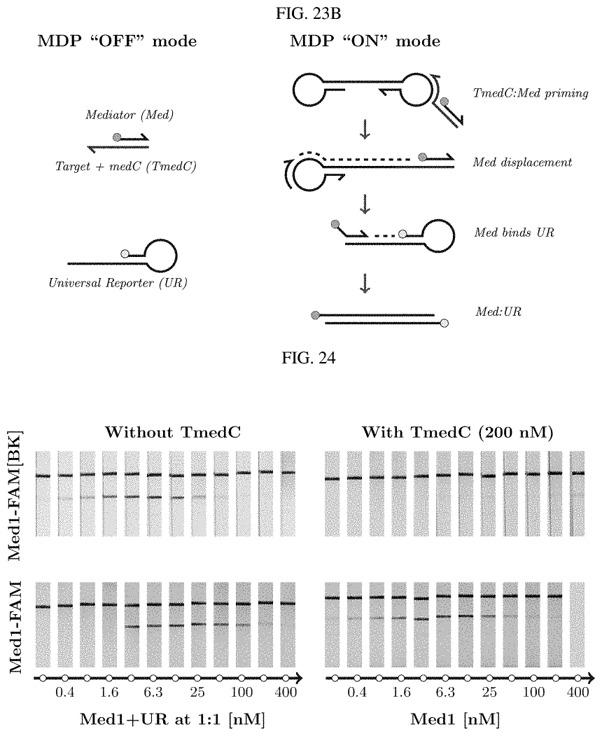

Mediator Displacement Probes (MDPs) were originally described as a method for detecting LAMP products using fluorescence. The strategy behind previously-used MDPs is illustrated and explained in FIG. 23A. Aspects of the disclosure relate to adapting MDPs for LFA, for example explained in FIG. 23B. Briefly, the modified methods work by using a primer that targets one the loop arms (either the forward or reverse loop), with an extension on the 5′ end (MedC sequence) that is annealed to its complementary sequence (Med sequence). The Med oligo should contain one of the desired labels for LFA detection. The Med oligo is released by enzymatic strand-displacement during LAMP, and primes into a second oligo also labelled for LFA (called Universal Reporter), generating a dually labelled molecule ready to be detected. The probes are added in addition to the 6 oligos regularly used for LAMP, and some concentration of the target loop oligo is replaced by T_MedC.

The advantages of the MDP strategy include: simple design (e.g., once established the functionality of the Med and UR probes, all that is left to design is the appropriate target sequence for the T_MedC primer and potentially calibrate its concentration), potential real-time optimization with fluorescent probes: It is possible to combine LFA and fluorescent probes into the same module, allowing real time visualization of dually-labelled molecules, and no loss in speed and sensitivity compared to regular LAMP: The chemistry from regular LAMP assays is expected to be transferable to MDP. The strategy sits on top of the regular LAMP chemistry and typical probe concentrations are within one to two order of magnitude lower.

Furthermore, the previously-used MDPs were improved in the context of LFA detection by blocking them to reduce background LFA signal and allow or direct application to LFA without post-amplification dilution. For a molecular diagnostic to be accurate, it must only generate a positive signal in the presence of the true molecular target. However, one of the early lessons we learned is that MDP are prone to generating LFA signal even in the absence of the target amplicon. This is perhaps not very surprising considering sensitivity of LFA detection may be different from fluorescence detection, the application for which the MDP probes were originally designed.

Background signal reduction was investigated by blocking the 3′-end of the Med probe, and varying the ratio of TmedC to Med probes to minimize free labeled strand in the system prior to amplification. However a high TmedC:Med ratio could impact sensitivity as a larger fraction of TmedC may be needed to be incorporated to generate sufficiently dually labeled molecules. Therefore, blocking the 3′-end was deemed to be the more favorable approach. The principle behind blocking Med 3′-end on background signal can be observed in FIG. 24, where Med1-FAM probes were titrated in the absence of primers while maintaining As1e_TmedC1 probes constant. As can be seen from the FIG. 24, the background signal is significantly higher in the case of free 3′-end. This is because the free 3′-end allows the DNA polymerase to extend the Med strand increasing the stability of the Med:UR complex, i.e the dually labelled molecule. In addition, it is worth noting that the observed concentration dependency is precisely due to the “hook” effect arising from saturation of the LFA antibodies.

### Example 4: Detection of Human RNAse P Using Med2/UR2 Probes

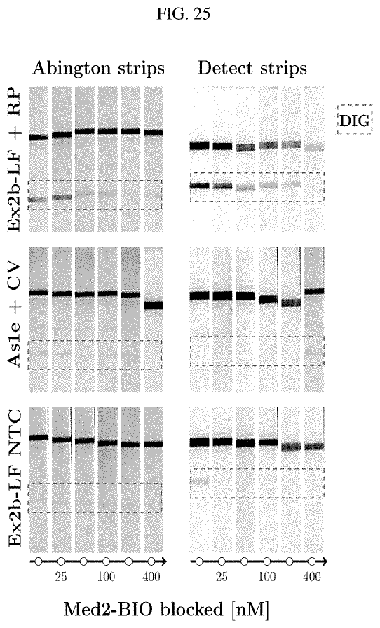

Med2 probes were evaluated for the detection of human RNAse P with direct application of the reacted NAA mix to LFA with no intervening steps or use of LFA running buffer. Similarly to Med1 probes, background signal was also a challenge. Nonetheless, a successful proof of concept after titrating the Med2 concentrations was achieved. FIG. 25 shows representative data for detection of Ex2b (RNAse P) amplicon using a Ex2b_LF/Med2-BIO[BK] probe. Ex2b_LF_Med2 probe and a Med2-BIO blocked probe were titrated in the presence of Ex2b primer set (minus LF) amplification and As1e (Sars-CoV-2) amplification. The TmedC/Med ratio was kept constant at 2:1 ratio. UR-DIG was also constant at 10 nM. Data indicate that near the 50-100 nM Med2 range, specific signal generation was produced on LFA strips. Two different LFA strip types were tested for completeness—“Abington” strips and “Detect” strips. Abington (lot E01) and Detect strips have different sensitivities to “background” LFA signal. On Abington, faint bands for both conjugates (FAM and DIG) can be observed regardless of probe concentrations. One possibility is that this effect is related to loading the undiluted LAMP buffer into the strip, as previous experimentation (not shown here) suggested that Tween-20 in the LAMP buffer could be responsible for these “faint” bands.

TABLE 3
RNAas P primer set

| Name | Sequence [5′-3′] | SEQ ID |
|---|---|---|
| Ex2b-FIP | TGTCGGTCTCTGGCTCCAC | 22 |
| | AGGTGGCTGCCAATAC | |
| Ex2b-BIP | CACGGGAGCCACTGACTCC | 23 |
| | CCTGAAGACTCGGATGT | |
| Ex2b-F3 | GCAGCTTCGGGTCCT | 24 |
| Ex2b-B3 | GGATAAGTGGAGGAGTGTCT | 25 |
| Ex2b-LF | TCAACAAGCTCCACGGTG | 26 |
| Ex2b-LB | TCCGCAACAACTCAGCC | 27 |

TABLE 4
SARS-CoV-2 primer set

| Name | Sequence [5′-3′] | SEQ ID |
|---|---|---|
| Asle-FIP | TCAGCACACAAAGCCAAAA | 28 |
| | ATTTATTTTTCTGTGC | |
| | AAAGGAAATTAAGGAG | |
| Asle-BIP | TATTGGTGGAGCTAAACT | 29 |
| | TAAAGCCTTTTCTGTAC | |
| | AATCCCTTTGAGTG | |
| Asle-F3 | CGGTGGACAAATTGTCAC | 30 |
| Asle-B3 | CTTCTCTGGATTTAACACA | 31 |
| | CTT | |
| Asle-LF | TTACAAGCTTAAAGAATGT | 32 |
| | CTGAACACT | |
| Asle-LB | TTGAATTTAGGTGAAACAT | 33 |
| | TTGTCACG | |

TABLE 5
Modified Mediator Displacement Probes

| | Top Strand | |
|---|---|---|
| | Bottom Strand | |
| Name | Sequence [5′-3′] | SEQ ID |
| COVID- | GGTCGTAGAGCCCAT | 34 |
| MedC₁ | TGCGCGATGAGTGG- | |
| | COVID_Primer | |
| RNAse | GGTCGTAGAGCCCAT | 35 |
| P-MedC₁ | TGCGCGATGAGTGG- | |
| | RNAseP_Primer | |
| COVID- | CACTGACCGAACTGA | 36 |
| MedC₂ | GCTCCTGAGGCATGG- | |
| | COVID_Primer | |
| RNAsc | CACTGACCGAACTGA | 37 |
| P-MedC₂ | GCTCCTGAGGCATGG- | |
| | RNAseP_Primer | |
| Med1 | CCACTCATCGCGCAA | 38 |
| | TGGGCTCTACGACC | |
| Med1 | CCACTCATCGCGCAA | 39 |
| [BK] | TGGGCTCTACGACC | |
| | [BK] | |
| Med2 | CCATGCCTCAGGAGC | 40 |
| | TCAGTTCGGTCAGTG | |
| | [BK] | |
| Med2[BK] | CCATGCCTCAGGAGC | 41 |
| | TCAGTTCGGTCAGTG | |
| | [BK] | |
| Universal | OptionalQuencher- | 42, 104 |
| Reporter 1 | ATTGCGGGAGATGAGA | |
| | CCCGCAA-dT- | |
| | HAPTEN-TGTTGGTC | |
| | GTAGAGCCCAGAACG | |
| | A-C3 | |
| Universal | OptionalQuencher- | 43, 105 |
| Reporter 2 | CACCGGCCAAGACGCG | |
| | CCGG-dT-HAPTEN- | |
| | GTGTTCACTGACCGA | |
| | ACTGGAGCA-C3 | |

### Example 5 Exemplary Reaction Protocol

Experiments were carried out by mixing a buffer twice as concentrated (2× buffer—pH 8.05 at 25° C.), either a lyo-bead or a frozen mix carrying all enzymes and nucleotides. Primers, probes and RNA templates were prepared in water and mixed subsequently. TmedC and Med probes were mixed in water at room temperature, and UR probes were added always right at the end before setting the reaction. All reactions were performed at 80 μl volume, and incubated in a PCR thermocycler following 3 mins at 37° C., 38 mins at 63.5° C., and held at 37° C. All LFA tests were performed using vertically inverted strips placed into 70-80 μl of liquid. The concentrations of all primers and probes were calibrated using OD260 measurements with a Nanodrop instrument.

In some embodiments, RT-LAMP chemistry conditions are as follows in any of Tables 6-10. In some embodiments, one or more primers listed in Tables 7-10 are used in a reaction buffer comprising the components listed in Table 6. In some embodiments, the reaction mixture further comprises a biological sample (or nucleic acids isolated from a biological sample, for example human nucleic acids and nucleic acids derived from a pathogen).

TABLE 6
Reaction Buffer

| Component | Concentration | |
|---|---|---|
| Tris-base | 20 | mM |
| (NH4)2SO4 | 10 | mM |
| Tween ® 20 | 0.1% | |
| KCl | 50 | mM |
| MgS04 | 8 | mM |
| EGTA | 200 | uM |
| dATP | 1.4 | mM |
| dCTP | 1.4 | mM |
| dGTP | 1.4 | mM |
| dTTP | 0.7 | mM |
| dUTP | 0.7 | mM |
| UDG - Thermolabile (ArcticZymes) | 0.02 | U/ul |
| Murine RI (NEB) | 0.5 | U/ul |
| RTx (NEB) | 0.3 | U/ul |
| WS Bst 2.0 (NEB) | 0.32 | U/ul |

TABLE 7
Ex2b primer set. Concentrations listed
represent final concentrations only when
they are used in reaction buffer.

| | | Con- | | |
|---|---|---|---|---|
| | | cen- | | |
| | | tra- | | |
| | | tion | | SEQ |
| Name | LIMS_ID | [nM] | Sequence [5′-3′] | ID |
| Ex2b-FIP | HD_ | 640 | TGTCGGTCTCTGGCTCCA | 44 |
| | 20.00829 | | CAGGTGGCTGCCAATAC | |
| Ex2b-BIP | HD_ | 640 | CACGGGAGCCACTGACTC | 45 |
| | 20.00830 | | CCCTGAAGACTCGGATGT | |
| Ex2b-F3 | HD_ | 80 | GCAGCTTCGGGTCCT | 46 |
| | 20.00827 | | | |
| Ex2b-B3 | HD_ | 80 | GGATAAGTGGAGG | 47 |
| | 20.00828 | | AGTGTCT | |
| Ex2b-LF- | HD_ | 160 | /5DigN/TCAACAAGC | 48 |
| DIG | 20.00833 | | TCCACGGTG | |
| Ex2b-LB- | HD_ | 160 | /5Biosg/TCCGCAAC | 49 |
| BIO | 20.00834 | | AACTCAGCC | |
| Ex2b-LF | HD_ | 160 | TCAACAAGCTC | 50 |
| | 20.00831 | | CACGGTG | |
| Ex2b-LB | HD_ | 160 | TCCGCAACAACTCAGCC | 51 |
| | 20.00832 | | | |

TABLE 8
Asle primer set. Concentrations listed
represent final concentrations only
when they are used in reaction buffer.

| | | Con- | | |
|---|---|---|---|---|
| | | cen- | | |
| | | tra- | | |
| | | tion | Sequence | SEQ |
| Name | LIMS_ID | [nM] | [5′-3′] | ID |
| Asle-FIP | HD_20.00689 | 1600 | TCAGCACACAAAGCCA | 52 |
| | | | AAAATTTATTTTTCTG | |
| | | | TGCAAAGGAAATTAAG | |
| | | | GAG | |
| Asle-BIP | HD_20.00692 | 1600 | TATTGGTGGAGCTAAA | 53 |
| | | | CTTAAAGCCTTTTCTG | |
| | | | TACAATCCCTTTGAGT | |
| | | | G | |
| Asle-F3 | HD_20.00676 | 200 | CGGTGGACAAATTGTC | 54 |
| | | | AC | |
| Asle-B3 | HD_20.00677 | 200 | CTTCTCTGGATTTAAC | 55 |
| | | | ACACTT | |
| Asle-LF- | HD_20.00685 | 400 | /56-FAM/TTACAA | 56 |
| FAM | | | GCTTAAAGAATGTC | |
| | | | TGAACACT | |
| Asle-LB- | HD_20.00682 | 400 | /5Biosg/TTGAATT | 57 |
| BIO | | | TAGGTGAAACATTTG | |
| | | | TCACG | |
| Asle-LF | HD_20.00680 | 400 | TTACAAGCTTAAAGA | 58 |
| | | | ATGTCTGAACACT | |
| Asle-LB | HD_20.00681 | 400 | TTGAATTTAGGTGAA | 59 |
| | | | ACATTTGTCACG | |

TABLE 9
Exemplary sequences of the Med1,
Med1b and Med2 pairs. Note homologous
pairings are underlined.

| | Med | UR | SEQ ID |
|---|---|---|---|
| | Sequence | Sequence | (L to |
| Pair | (5′-3′) | (5′-3′) | R) |
| Med1/ | CCACTCATCGCG | ATTGCGGGAGATGA | 60, 61 |
| UR1 | CAATGGGCTCT | GACCCGCAATTG | |
| | ACGACC | TTGGTCGTAGAGC | |
| | | CCAGAACGA | |
| Med1b/ | CCACTCATCTCG | ATTGCGGGAGATGAG | 62, 63 |
| UR1 | TTCTGGGCTCT | ACCCGCAATTGTTG | |
| | ACGACC | GTCGTAGAGCCCAG | |
| | | AACGA | |
| Med2/ | CCATGCCTCAGG | CACCGGCCAAGACGC | 64, 65 |
| UR2 | AGCTCAGTTCGGT | GCCGGTGTGTTCACT | |
| | CAGTG | GACCGAACTGGAGCA | |

TABLE 10
Mediator displacement probes for LFA detection.
Probes listed here in alphabetical order. Note
that by convention T_MedC probes are named
first by primer set target, then by loop
(either forward or reverse), and then
by complementary sequence to Med probe.

| | LIMS_ | Sequence | SEQ |
|---|---|---|---|
| Name | ID | [5′-3′] | ID |
| Asle_LB_ | HD_ | GGTCGTAGAGCCCAT | 66 |
| MedC1 | 20.01078 | TGCGCGATGAGTGGt | |
| | | tgaatttaggtgaaa | |
| | | catttgtcacg | |
| Asle_LB_ | HD_ | GGTCGTAGAGCCCAG | 67 |
| MedC1b | 20.0109 | AACGAGATGAGTGGT | |
| | | TG | |
| | | AATTTAGGTGAA | |
| | | ACATTTGTCACG | |
| Asle_LB_ | HD_ | CACTGACCGAACTGA | 68 |
| MedC2 | 20.0108 | GCTCCTGAGGCATGG | |
| | | ttgaatttaggtgaa | |
| | | acatttgtcacg | |
| Asle_LF_ | HD_ | GGTCGTAGAGCCCAT | 69 |
| MedC1 | 20.01001 | TGCGCGATGAGTGGT | |
| | | TACAAGCTTAAAGA | |
| | | ATGTCTGAACACT | |
| Asle_LF_ | HD_ | GGTCGTAGAGCCCAG | 70 |
| MedC1b | 20.01089 | AACGAGATGAGTGGT | |
| | | TACAAGCTTAAAGA | |
| | | ATGTCTGAACACT | |
| Asle_LF_ | HD_ | CACTGACCGAACTGA | 71 |
| MedC2 | 20.01082 | GCTCCTGAGGCATGG | |
| | | TTACAAGCTTAAAG | |
| | | AATGTCTGAACACT | |
| Ex2b_LB_ | HD_ | GGTCGTAGAGCCCAT | 72 |
| MedC1 | 20.01081 | TGCGCGATGAGTGGt | |
| | | ccgcaacaactcag | |
| | | cc | |
| Ex2b_LB_ | HD_ | GGTCGTAGAGCCCAG | 73 |
| MedC1b | 21.00025 | AACGAGATGAGTGGt | |
| | | ccgcaacaactcagc | |
| | | c | |
| Ex2b_LB_ | HD_ | CACTGACCGAACTGA | 74 |
| MedC2 | 20.01079 | GCTCCTGAGGCATGG | |
| | | tccgcaacaactca | |
| | | gcc | |
| Ex2b_LF_ | HD_ | GGTCGTAGAGCCCAT | 75 |
| MedC1 | 20.01084 | TGCGCGATGAGTGGT | |
| | | CAACAAGCTCCACGG | |
| | | TG | |
| Ex2b_LF_ | HD_ | GGTCGTAGAGCCCAG | 76 |
| MedC1b | 21.00024 | AACGAGATGAGTGGT | |
| | | CAACAAGCTCCACG | |
| | | GTG | |
| Ex2b_LF_ | HD_ | CACTGACCGAACTGA | 77 |
| MedC2 | 20.01072 | GCTCCTGAGGCATGG | |
| | | TCAACAAGCTCCAC | |
| | | GGTG | |
| Med1_3BIO | HD_ | CCACTCATCGCGCAA | 78 |
| | 21.00169 | TGGGCTCTACGACC/ | |
| | | 3Bio/ | |
| Med1_3DIG | HD_ | CCACTCATCGCGCAA | 79 |
| | 21.00168 | TGGGCTCTACGACC/ | |
| | | 3Dig_N/ | |
| Med1_BIO | HD_ | /5Biosg/CCACTCA | 80 |
| | 20.01005 | TCGCGCAATGGGCTC | |
| | | TACGACC | |
| Med1_BIO | HD_ | /5Biosg/CCACTCA | 81 |
| [BK] | 20.01006 | TCGCGCAATGGGCTC | |
| | | TACGACC/3InvdT/ | |
| Med1_DIG | HD_ | /5DigN/CCACTCAT | 82 |
| | 21.00006 | CGCGCAATGGGCTCT | |
| | | ACGACC | |
| Med1_FAM | HD_ | /56-FAM/CCACTCA | 83 |
| | 21.00001 | TCGCGCAATGGGCTC | |
| | | TACGACC | |
| Med1_FAM | HD_ | CCACTCATCGCGCAA | 84 |
| [b1ocked] | 20.01002 | TGGGCTCTACGACC/ | |
| | | 36-FAM/ | |
| Med1b_3BIO | HD_ | CCACTCATCTCGTTC | 85 |
| | 21.00172 | TGGGCTCTACGACC/ | |
| | | 3Bio/ | |
| Med1b_3DIG | HD_ | CCACTCATCTCGTTC | 86 |
| | 21.00171 | TGGGCTCTACGACC/ | |
| | | 3Dig_N/ | |
| Med1b_3FAM | HD_ | CCACTCATCTCGTTC | 87 |
| | 21.0017 | TGGGCTCTACGACC/ | |
| | | 36-FAM/ | |
| Med1b_BIO | HD_ | /5Biosg/CCACTCA | 88 |
| | 20.01088 | TCTCGTTCTGGGCTC | |
| | | TACGACC | |
| Med1b_DIG | HD_ | /5DigN/CCACTCAT | 89 |
| | 21.0003 | CTCGTTCTGGGCTCT | |
| | | ACGACC | |
| Med1b_FAM | HD_ | /56-FAM/CCACTCA | 90 |
| | 21.00002 | TCTCGTTCTGGGCTC | |
| | | TACGACC | |
| Med2_3BIO | HD_ | CATGCCTCAGGAGCT | 91 |
| | 21.00175 | CAGTTCGGTCAGTG/ | |
| | | 3Bio/ | |
| Med2_3DIG | HD_ | CATGCCTCAGGAGCT | 92 |
| | 21.00174 | CAGTTCGGTCAGTG/ | |
| | | 3Dig_N/ | |
| Med2_3FAM | HD_ | CATGCCTCAGGAGCT | 93 |
| | 21.00173 | CAGTTCGGTCAGTG/ | |
| | | 36-FAM/ | |
| Med2_BIO | HD_ | /5Biosg/CCATGCC | 94 |
| | 20.01073 | TCAGGAGCTCAGTTC | |
| | | GGTCAGTG | |
| Med2_BIO | HD_ | /5Biosg/CCATGCC | 95 |
| [b1ocked] | 20.01074 | TCAGGAGCTCAGTTC | |
| | | GGTCAGTG/ | |
| | | 3InvdT/ | |
| Med2_DIG | HD_ | /5DigN/CCATGCCT | 96 |
| | 21.00007 | CAGGAGCTCAGTTCG | |
| | | GTCAGTG | |
| Med2_FAM | HD_ | /56-FAM/CCATGCC | 97 |
| | 21.00003 | TCAGGAGCTCAGTTC | |
| | | GGTCAGTG | |
| UR1_BIO | HD_ | ATTGCGGGAGATGAG | 98 |
| | 21.00004 | ACCCGCAAtTGTTGG | |
| | | TCGTAGAGCCCAGAA | |
| | | CGA/3Bio/ | |
| UR1_DIG | HD_ | ATTGCGGGAGATGAG | 99 |
| | 21.00008 | ACCCGCAAtTGTTGG | |
| | | TCGTAGAGCCCAGAA | |
| | | CGA/3Dig_N/ | |
| UR1_FAM | HD_ | ATTGCGGGAGATGAG | 100 |
| | 20.01116 | ACCCGCAAtTGTTGG | |
| | | TCGTAGAGCCCAGAA | |
| | | CGA/36-FAM/ | |
| UR2_BIO | HD_ | CACCGGCCAAGACGC | 101 |
| | 21.00005 | GCCGGtGTGTTCACT | |
| | | GACCGAACTGGAGCA/ | |
| | | 3Bio/ | |
| UR2_DIG | HD_ | CACCGGCCAAGACGC | 102 |
| | 21.00009 | GCCGGtGTGTTCACT | |
| | | GACCGAACTGGAGCA/ | |
| | | 3Dig_N/ | |
| UR2_FAM | HD_ | CACCGGCCAAGACGC | 103 |
| | 20.01117 | GCCGGtGTGTTCACT | |
| | | GACCGAACTGGAGCA/ | |
| | | 36-FAM/ | |

### ADDITIONAL EMBODIMENTS

Embodiment 1: A method for detecting target nucleic acids in a biological sample, the method comprising:

(i) performing an isothermal amplification reaction on a biological sample comprising a target gene and a control gene,

wherein the amplification reaction is performed in a buffer solution comprising one or more biotinylated oligonucleotides, a target nucleic acid-specific primer comprising a first hapten, and a control nucleic acid-specific primer comprising a second hapten different from the first hapten, wherein the buffer comprises a total concentration of biotinylated oligonucleotides that is less than about 200 nM, a total concentration of first hapten less than or equal to about 25 nM, and a total concentration of second hapten that is less than or equal to about 100 nM, thereby producing an amplification product mixture comprising labeled target amplification products and labeled control amplification products;

(ii) contacting a lateral flow assay (LFA) device comprising a first test line that specifically binds to the first hapten and a second test line that specifically binds to the second hapten with an undiluted aliquot of the amplification product mixture under conditions under which the labeled target amplification products bind to the first test line to produce a first detectable signal or the labeled control amplification products bind to the second test line to produce a second detectable signal; and

(iii) identifying the presence of the target nucleic acid in the biological sample based upon detecting the presence of the first detectable signal and the second detectable signal, or identifying the absence of the target nucleic acid in the biological sample based on detecting the absence of the first detectable signal and the presence of the second detectable signal.

Embodiment 2: The method of embodiment 1, wherein the target nucleic acid comprises DNA or RNA.
Embodiment 3: The method of embodiment 1 or 2, wherein the target nucleic acid is derived from a pathogen.
Embodiment 4: The method of embodiment 3, wherein the pathogen is a viral, bacterial, fungal, parasitic, or protozoan pathogen.
Embodiment 5: The method of any one of embodiments 1 to 4, wherein the biological comprises blood, saliva, mucous, urine, feces, cerebrospinal fluid (CSF), or tissue.
Embodiment 6: The method of any one of embodiments 1 to 5, wherein the biological sample is obtained from a subject.
Embodiment 7: The method of embodiment 6, wherein the subject is a mammal, optionally wherein the subject is a human.
Embodiment 8: The method of any one of embodiments 1 to 7, wherein the control nucleic acid is derived from a different species than the target nucleic acid.
Embodiment 9: The method of embodiment 8, wherein the control nucleic acid is derived from a human.
Embodiment 10: The method of any one of embodiments 1 to 9, wherein the isothermal amplification reaction comprises loop-mediated isothermal amplification (LAMP), optionally wherein the LAMP comprises reverse transcription LAMP (RT-LAMP).
Embodiment 11: The method of any one of embodiments 1 to 10, wherein the isothermal amplification reaction is performed using a polymerase selected from the group consisting of Bacillus stearothermophilus (Bst), Bacillus Smithii (Bsm), Geobacillus sp. M (GspM), Thermodesulfatator indicus (Tin), and Taq DNA polymerase.
Embodiment 12: The method of embodiment 10 or 11, wherein the reverse transcriptase used for RT-LAMP is selected from the group consisting of HIV-1 reverse transcriptase, Moloney murine leukemia virus (M-MLV) reverse transcriptase, and avian myeloblastosis virus (AMV) reverse transcriptase.
Embodiment 13: The method of any one of embodiments 1 to 12, wherein the one or more biotinylated oligonucleotides comprise a target nucleic acid-specific biotinylated primer and/or a control nucleic acid-specific biotinylated primer.
Embodiment 14: The method of any one of embodiments 1 to 13, wherein the first hapten comprises a Fluorescein isothiocyanate (FITC) molecule.
Embodiment 15: The method of embodiment 14, wherein the concentration of the first hapten in the buffer solution ranges from about 5 nM to about 25 nM.
Embodiment 16: The method of any one of embodiments 1 to 15, wherein the second hapten comprises a digoxigenin molecule.
Embodiment 17: The method of embodiment 16, wherein the concentration of the second hapten in the buffer solution ranges from about 5 nM to about 100 nM.
Embodiment 18: The method of any one of embodiments 14 to 17, wherein the target nucleic acid-specific biotinylated primer and the control nucleic acid-specific biotinylated primer are present in non-equimolar concentrations in the buffer solution.
Embodiment 19: The method of embodiment 18, wherein the ratio of concentration of the target nucleic acid-specific biotinylated primer to the control nucleic acid-specific biotinylated primer ranges from about 1:5 to about 5:1.
Embodiment 20: The method of embodiment 18, wherein the ratio of concentration of the target nucleic acid-specific biotinylated primer to the control nucleic acid-specific biotinylated primer ranges from about 1:3 to about 3:1.
Embodiment 21: The method of any one of embodiments 1 to 20, wherein the buffer solution further comprises one or more sets of outer primers.
Embodiment 22: The method of embodiment 21, wherein at least one set of outer primers specifically binds to the target nucleic acid.
Embodiment 23: The method of embodiment 21 or 22, wherein at least one set of outer primers specifically binds to the control nucleic acid.
Embodiment 24: The method of any one of embodiments 1 to 23, wherein the buffer solution further comprises one or more sets of internal primers.
Embodiment 25: The method of embodiment 24, wherein at least one set of internal primers specifically binds to the target nucleic acid.
Embodiment 26: The method of embodiment 24, wherein at least one set of internal primers specifically binds to the control nucleic acid.
Embodiment 27: The method of any one of embodiments 1 to 26, wherein the amplification reaction proceeds for less than 45 minutes.
Embodiment 28: The method of any one of embodiments 1 to 27, wherein the identifying comprises visually confirming the presence of the first detectable signal and/or the second detectable signal.
Embodiment 29: The method of any one of embodiments 1 to 28 further comprising identifying the subject as being infected with a pathogen based upon the presence of the target nucleic acid.
Embodiment 30: The method of embodiment 29 further comprising administering one or more therapeutic agents to the subject.
Embodiment 31: A method for amplifying a target nucleic acid, the method comprising:

(i) contacting a target nucleic acid in biological sample obtained from a subject with an amplification buffer to produce an amplification mixture, wherein the amplification buffer comprises:
(a) one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.);
(b) an outer primer pair comprising a region of complementarity with the target nucleic acid;
(c) an internal primer pair comprising a region of complementarity with the target nucleic acid;
(d) a first loop primer pair comprising a first loop primer linked to a biotin, a second loop primer linked to a first hapten, wherein the first and second loop primers each comprise a region of complementarity with the target nucleic acid, and wherein the concentration of the first loop primer in the amplification mixture is less than or equal to 200 nM and the concentration of the second loop primer in the amplification mixture is less than or equal to 100 nM;
(e) a deoxynucleoside triphosphate (dNTP) mixture;
(e) a polymerase; and
(f) a reverse transcriptase; and

(ii) performing an isothermal amplification reaction on the amplification mixture to produce a composition comprising an increased amount of target nucleic acid relative to the amount of target nucleic acid in the biological sample.

Embodiment 32: The method of embodiment 31, wherein the target nucleic acid is DNA or RNA.
Embodiment 33: The method of embodiment 31 or 32, wherein the biological sample comprises blood, saliva, mucous, urine, feces, cerebrospinal fluid (CSF), or tissue.
Embodiment 34: The method of any one of embodiments 31 to 33, wherein the biological sample further comprises a control nucleic acid and the amplification buffer further comprises:
(g) a second outer primer pair comprising a region of complementarity with the control nucleic acid;
(h) a second internal primer pair comprising a region of complementarity with the control nucleic acid; and/or
(i) a second loop primer pair comprising a third loop primer linked to a biotin and a fourth loop primer linked to a second hapten, wherein the third and fourth loop primers each comprise a region of complementarity with the control nucleic acid.
   Embodiment 35: The method of any one of embodiments 31 to 34, wherein the first hapten comprises FITC.
   Embodiment 36: The method of embodiment 34 or 35, wherein the second hapten comprises digoxigenin.
   Embodiment 37: The method of any one of embodiments 34 to 36, wherein the first loop primer and the third loop primer are present in non-equimolar concentrations in the amplification buffer.
   Embodiment 38: The method of any one of embodiments 31 to 37, wherein the concentration of the second loop primer in the amplification buffer ranges from about 5 nM to about 25 nM.
   Embodiment 39: The method of any one of embodiments 34 to 38, wherein the concentration of the fourth loop primer in the amplification buffer ranges from about 5 nM to about 100 nM.
   Embodiment 40: The method of any one of embodiments 31 to 39, wherein the dNTP mixture comprises deoxyadenosine 5′-triphosphate (dATP), deoxyguanine 5′-triphosphate (dGTP), deoxythymidine 5′-triphosphate (dTTP), deoxycytidine 5′-triphosphate (dCTP), and 2′-Deoxyuridine, 5′-Triphosphate (dUTP).
   Embodiment 41: The method of any one of embodiments 31 to 40, wherein the polymerase is selected from the group consisting of Bacillus stearothermophilus (Bst), Bacillus Smithii (Bsm), Geobacillus sp. M (GspM), Thermodesulfatator indicus (Tin), and Taq DNA polymerase.
   Embodiment 42: The method of any one of embodiments 31 to 41, wherein the reverse transcriptase is selected from the group consisting of HIV-1 reverse transcriptase, Moloney murine leukemia virus (M-MLV) reverse transcriptase, and avian myeloblastosis virus (AMV) reverse transcriptase.
   Embodiment 43: The method of any one of embodiments 31 to 42, wherein the isothermal amplification reaction comprises loop-mediated isothermal amplification (LAMP), optionally wherein the LAMP comprises reverse transcription LAMP (RT-LAMP).
   Embodiment 44: The method of any one of embodiments 31 to 43, wherein the target nucleic acid is derived from a pathogen.
   Embodiment 45: The method of embodiment 44, wherein the pathogen is a viral, bacterial, fungal, parasitic, or protozoan pathogen.
   Embodiment 46: The method of embodiment 45, wherein the viral pathogen is SARS-CoV-2.
   Embodiment 47: The method of any one of embodiments 34 to 46, wherein the control nucleic acid is derived from a mammal, optionally a human.
   Embodiment 48: A method for preparing a population of nucleic acid amplification products useful for analyzing a target gene in a biological sample obtained from a subject, the method comprising:

(i) performing an isothermal amplification reaction on a biological sample comprising a target gene and a control gene,

wherein the amplification reaction is performed in a buffer solution comprising one or more biotinylated oligonucleotides, a target nucleic acid-specific primer comprising a first hapten, and a control nucleic acid-specific primer comprising a second hapten different from the first hapten, wherein the buffer comprises a total concentration of biotinylated oligonucleotides that is less than 200 nM, a total concentration of first hapten less than or equal to 25 nM, and a total concentration of second hapten that is less than or equal to 100 nM, thereby producing an amplification product mixture comprising labeled target amplification products and labeled control amplification products; and

(ii) analyzing a target gene in the amplification product mixture produced in (i).

Embodiment 49: The method of embodiment 48, wherein the analyzing comprises contacting a lateral flow assay (LFA) device comprising a first test line that specifically binds to the first hapten and a second test line that specifically binds to the second hapten with an undiluted aliquot of the amplification product mixture under conditions under which the labeled target amplification products bind to the first test line to produce a first detectable signal or the labeled control amplification products bind to the second test line to produce a second detectable signal.
Embodiment 50: The method of embodiment 48 or 49, wherein the analyzing further comprises identifying the presence of the target nucleic acid in the biological sample based upon detecting the presence of the first detectable signal and the second detectable signal, or identifying the absence of the target nucleic acid in the biological sample based on detecting the absence of the first detectable signal and the presence of the second detectable signal.
Embodiment 51: The method of embodiment 50 further comprising the step of identifying the subject from which the biological sample was obtained as being infected with an organism encoding the target nucleic acid when the target nucleic acid is present in the amplification product mixture.
Embodiment 52: An amplification mixture comprising:
(a) one or more buffering agents (e.g., Tris-HCl, tricine, bicine, (NH₄)2SO₄, etc.), one or more salts (e.g., KCl, MgSO₄, etc.) and one or more detergents (e.g., Tween20, Triton-X-100, etc.);
(b) an outer primer pair comprising a region of complementarity with the target nucleic acid;
(c) an internal primer pair comprising a region of complementarity with the target nucleic acid;
(d) a first loop primer pair comprising a first loop primer linked to a biotin and a second loop primer linked to a first hapten, wherein the first and second loop primers each comprise a region of complementarity with the target nucleic acid, and wherein the concentration of the first loop primer in the amplification mixture is less than or equal to 200 nM and the concentration of the second loop primer in the amplification mixture is less than or equal to 100 nM;
(e) a deoxynucleoside triphosphate (dNTP) mixture;
(e) a polymerase; and
(f) a reverse transcriptase.
   Embodiment 53: The amplification mixture of embodiment 52 further comprising a biological sample.
   Embodiment 54: The amplification mixture of embodiment 53, wherein the biological sample comprises blood, saliva, mucous, urine, feces, cerebrospinal fluid (CSF), or tissue.
   Embodiment 55: The amplification mixture of embodiment 53 or 54, wherein the biological sample comprises a target nucleic acid.
   Embodiment 56: The amplification mixture of embodiment 55, wherein the target nucleic acid is derived from a pathogen.
   Embodiment 57: The amplification mixture of embodiment 56, wherein the pathogen is a viral, bacterial, fungal, parasitic, or protozoan pathogen.
   Embodiment 58: The amplification mixture of embodiment 57, wherein the viral pathogen is SARS-CoV-2.
   Embodiment 59: The amplification mixture of any one of embodiments 52 to 58 further comprising a control nucleic acid.
   Embodiment 60: The amplification mixture of embodiment 60, wherein the control nucleic acid is derived from a human.
   Embodiment 61: The amplification mixture of any one of embodiments 52 to 60 further comprising:
(g) a second outer primer pair comprising a region of complementarity with the control nucleic acid;
(h) a second internal primer pair comprising a region of complementarity with the control nucleic acid; and/or
(i) a second loop primer pair comprising a third loop primer linked to a biotin and a fourth loop primer linked to a second hapten, wherein the third and fourth loop primers each comprise a region of complementarity with the control nucleic acid.
   Embodiment 62: The amplification mixture of any one of embodiments 52 to 61, wherein the dNTP mixture comprises deoxyadenosine 5′-triphosphate (dATP), deoxyguanine 5′-triphosphate (dGTP), deoxythymidine 5′-triphosphate (dTTP), deoxycytidine 5′-triphosphate (dCTP), and 2′-Deoxyuridine, 5′-Triphosphate (dUTP).
   Embodiment 63: The amplification mixture of any one of embodiments 52 to 62, wherein the polymerase is selected from the group consisting of Bacillus stearothermophilus (Bst), Bacillus Smithii (Bsm), Geobacillus sp. M (GspM), Thermodesulfatator indicus (Tin), and Taq DNA polymerase.
   Embodiment 64: The amplification mixture of any one of embodiments 52 to 63, wherein the reverse transcriptase is selected from the group consisting of HIV-1 reverse transcriptase, Moloney murine leukemia virus (M-MLV) reverse transcriptase, and avian myeloblastosis virus (AMV) reverse transcriptase.
   Embodiment 65: A container comprising the amplification mixture of any one of embodiments 52 to 64.
   Embodiment 66: An oligonucleotide strand displacement probe comprising:
(i) a first single stranded polynucleotide having a 5′ portion and a 3′ portion, wherein the 5′ portion comprises a first hapten and the 3′ portion comprises an extension blocking group; and
(ii) a second single stranded polynucleotide comprising a 5′ portion and a 3′ portion, wherein the 5′ portion comprises a biotin,
   wherein the second single stranded polynucleotide is hybridized to the first single stranded polynucleotide to form a duplex region and a single stranded toehold region comprising the 3′ portion of the first single stranded polynucleotide.
   Embodiment 67: The oligonucleotide strand displacement probe of embodiment 66, wherein the first single stranded polynucleotide ranges from about 10 to about 50 nucleotides in length.
   Embodiment 68: The oligonucleotide strand displacement probe of embodiment 66, wherein the first second stranded polynucleotide ranges from about 10 to about 50 nucleotides in length.
   Embodiment 69: The oligonucleotide strand displacement probe of any one of embodiments 66 to 68, wherein the duplex region ranges from about 5 nucleotides to about 40 nucleotides in length.
   Embodiment 70: The oligonucleotide strand displacement probe of any one of embodiments 66 to 68, wherein the duplex region comprises a blunt end.
   Embodiment 71: The oligonucleotide strand displacement probe of any one of embodiments 66 to 70, wherein the single stranded toehold region ranges from about 5 to about 45 nucleotides in length.
   Embodiment 72: The oligonucleotide strand displacement probe of any one of embodiments 66 to 71, wherein the single stranded toehold region comprises a region of complementarity with a target gene.
   Embodiment 73: The oligonucleotide strand displacement probe of any one of embodiments 66 to 72, wherein the target gene is derived from a pathogen.
   Embodiment 74: The oligonucleotide strand displacement probe of embodiment 73, wherein the pathogen is a viral, bacterial, fungal, parasitic, or protozoan pathogen.
   Embodiment 75: The oligonucleotide strand displacement probe of embodiment 74, wherein the viral pathogen is SARS-CoV-2 Embodiment 76: The oligonucleotide strand displacement probe of any one of embodiments 66 to 75, wherein the first hapten comprises FITC.
   Embodiment 77: The oligonucleotide strand displacement probe of any one of embodiments 66 to 76, wherein the extension blocking group comprises a dehydroxylated 3′ nucleotide, 3′ dideoxycytosine (ddC), 3′ inverted deoxythymidine (dT), 3′ propyl (C3) spacer, 3′ amino modification, or 3′ phosphoryl modification.
   Embodiment 78: An amplification mixture comprising the oligonucleotide strand displacement probe of any one of embodiments 66 to 77 and one or more of the following:
(a) one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.);
(b) an outer primer pair comprising a region of complementarity with a target nucleic acid;
(c) an internal primer pair comprising a region of complementarity with the target nucleic acid;
(d) a first loop primer pair comprising a first loop primer linked to a biotin and a second loop primer linked to a first hapten, wherein the first and second loop primers each comprise a region of complementarity with the target nucleic acid;
(e) a deoxynucleoside triphosphate (dNTP) mixture;
(e) a polymerase; and
(f) a reverse transcriptase.
   Embodiment 79: An amplification mixture comprising:
(a) one or more buffering agents (e.g., Tris-HCl, tricine, bicine, (NH₄)₂SO₄, etc.), one or more salts (e.g., KCl, MgSO₄, etc.) and one or more detergents (e.g., Tween20, Triton-X-100, etc.);
(b) an outer primer pair comprising a region of complementarity with a target nucleic acid;
(c) an internal primer pair comprising a region of complementarity with the target nucleic acid;
(d) a first loop primer pair comprising a first loop primer linked to a biotin and a second loop primer linked to a first hapten, wherein the first and second loop primers each comprise a region of complementarity with the target nucleic acid;
(e) an oligonucleotide strand displacement probe comprising
   (i) a first single stranded polynucleotide having a 5′ portion and a 3′ portion, wherein the 3′ portion comprises an extension blocking group; and
   (ii) a second single stranded polynucleotide comprising a 5′ portion and a 3′ portion, wherein the 5′ portion comprises a biotin and the 3′ portion comprises an extension blocking group, and wherein the second single stranded polynucleotide is hybridized to the first single stranded polynucleotide to form a duplex region and a single stranded toehold region comprising the 3′ portion of the first single stranded polynucleotide;
(f) a single stranded reporter oligonucleotide that is complementary to the second single stranded polynucleotide of (e)(ii);
(e) a deoxynucleoside triphosphate (dNTP) mixture;
(e) a polymerase; and
(f) a reverse transcriptase.
   Embodiment 80: The amplification mixture of embodiment 79, wherein the 5′ portion of the first single stranded polynucleotide of (e)(i) comprises a digoxigenin (DIG).
   Embodiment 81: The amplification mixture of embodiment 79 or 80, wherein the single stranded reporter oligonucleotide comprises a FITC on its 5′ portion or 3′ portion.
   Embodiment 82: The amplification mixture of any one of embodiments 79 to 81, wherein the single stranded reporter oligonucleotide comprises an extension blocking group on its 5′ portion or 3′ portion.
   Embodiment 83: A method for detecting target nucleic acids in a biological sample, the method comprising:

(i) contacting the amplification mixture of embodiment 78 with a biological sample;

(ii) performing an isothermal amplification reaction on the mixture to produce an amplification mixture;

(iii) contacting the amplification mixture to a lateral flow assay (LFA) device under conditions under which the oligonucleotide strand displacement probe in the amplification mixture produces a first detectable signal when a target nucleotide is absent from the biological sample and/or a second detectable signal when a control nucleic acid is present in the biological sample;

(iii) identifying the presence of the target nucleic acid in the biological sample based upon detecting the absence of a first detectable signal and the presence of a second detectable signal, or identifying the absence of the target nucleic acid in the biological sample based on detecting the presence of the first detectable signal and the presence of the second detectable signal.

Embodiment 84: A method for detecting target nucleic acids in a biological sample, the method comprising:

(i) contacting the amplification mixture of any one of embodiments 79 to 82 with a biological sample;

(ii) performing an isothermal amplification reaction on the mixture to produce an amplification mixture;

(iii) contacting the amplification mixture to a lateral flow assay (LFA) device under conditions under which the single stranded reporter oligonucleotide in the amplification mixture produces a first detectable signal when a target nucleotide is present from the biological sample or a second detectable signal when the target nucleic acid is absent from the biological sample;

(iii) identifying the presence of the target nucleic acid in the biological sample based upon detecting the presence of a first detectable signal and the absence of a second detectable signal, or identifying the absence of the target nucleic acid in the biological sample based on detecting the absence of the first detectable signal and the presence of the second detectable signal.

Embodiment 85: The method of embodiment 83 or 84, wherein the biological sample comprises blood, saliva, mucous, urine, feces, cerebrospinal fluid (CSF), or tissue.
Embodiment 86: The method of any one of embodiments 83 to 85, wherein the target nucleic acid is derived from a pathogen.
Embodiment 87: The method of embodiment 87, wherein the pathogen is a viral, bacterial, fungal, parasitic, or protozoan pathogen.
Embodiment 88: The method of embodiment 87, wherein the viral pathogen is SARS-CoV-2.
Embodiment 89: The method of any one of embodiments 83 to 88 further comprising a step of detecting the presence of one or more control nucleic acids.
Embodiment 90: The method of embodiment 89, wherein the one or more control nucleic acids are derived from a human.

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

1. An oligonucleotide strand displacement probe comprising:
(i) a first single stranded polynucleotide having a 5′ portion and a 3′ portion, wherein the 3′ portion comprises a first hapten or the 5′ portion comprises a first hapten and the 3′ portion comprises a first extension blocking group; and
(ii) a second single stranded polynucleotide comprising a 5′ portion and a 3′ portion, wherein the 3′ portion comprises a second hapten or the 5′ portion comprises a second hapten and the 3′ portion comprises a second extension blocking group,
wherein the second single stranded polynucleotide is hybridized to the first single stranded polynucleotide to form a duplex region and a single stranded toehold region comprising the 3′ portion of the first single stranded polynucleotide.

2. The oligonucleotide strand displacement probe of claim 1, wherein the first single stranded polynucleotide ranges from about 10 to about 50 nucleotides in length.

3. The oligonucleotide strand displacement probe of claim 1, wherein the second stranded polynucleotide ranges from about 10 to about 50 nucleotides in length.

4. The oligonucleotide strand displacement probe of claim 1, wherein the single stranded toehold region comprises a region of complementarity with a target gene.

5. The oligonucleotide strand displacement probe of claim 1, wherein the target gene is derived from a pathogen.

6. The oligonucleotide strand displacement probe of claim 1, wherein the first hapten or the second hapten comprises FITC.

7. The oligonucleotide strand displacement probe of claim 1, wherein the first extension blocking group or second extension blocking group comprises a dehydroxylated 3′ nucleotide, 3′ dideoxycytosine (ddC), 3′ inverted deoxythymidine (dT), 3′ propyl (C3) spacer, 3′ amino modification, or 3′ phosphoryl modification.

8. An amplification mixture comprising the oligonucleotide strand displacement probe of claim 1, and one or more of the following:
(a) one or more buffering agents, one or more salts, and one or more detergents;
(b) an outer primer pair comprising a region of complementarity with a target nucleic acid;
(c) an internal primer pair comprising a region of complementarity with the target nucleic acid;
(d) a first loop primer pair comprising a first loop primer and a second loop primer, wherein the first and second loop primers each comprise a region of complementarity with the target nucleic acid;
(e) a deoxynucleoside triphosphate (dNTP) mixture;
(f) a polymerase; and
(g) a reverse transcriptase.

9. A method for detecting target nucleic acids in a biological sample, the method comprising:
(i) contacting the amplification mixture of claim 8 with a biological sample;
(ii) performing an isothermal amplification reaction on the mixture to produce an amplification mixture;
(iii) contacting the amplification mixture to a lateral flow assay (LFA) device under conditions under which the single stranded reporter oligonucleotide in the amplification mixture produces a first detectable signal when a target nucleotide is present from the biological sample or a second detectable signal when the target nucleic acid is absent from the biological sample;
(iii) identifying the presence of the target nucleic acid in the biological sample based upon detecting the presence of a first detectable signal and the absence of a second detectable signal, or identifying the absence of the target nucleic acid in the biological sample based on detecting the absence of the first detectable signal and the presence of the second detectable signal.

10. A mediator displacement probe comprising a hemi-duplex nucleic acid comprising:
(i) a first single stranded polynucleotide having a 5′ end and a 3′ end, comprising a target sequence-specific portion contiguously linked to a mediator complement (MedC) portion; and
(ii) a second single stranded polynucleotide having a 5′ end and a 3′ end, comprising a mediator (Med) polynucleotide sequence, a label attached to the 5′ end of the Med polynucleotide sequence, and an extension blocking agent attached to the 3′ end of the Med polynucleotide sequence,
wherein the MedC portion of the first single stranded polynucleotide and the Med polynucleotide sequence of the second single stranded polynucleotide are annealed together to form a hemi-duplex molecule, wherein the target sequence-specific portion of the first single stranded polynucleotide forms the single stranded portion of the hemi-duplex molecule.

11. The mediator displacement probe of claim 10, wherein the first single stranded polynucleotide ranges from about 10 to about 50 nucleotides in length.

12. The mediator displacement probe of claim 10, wherein the second stranded polynucleotide ranges from about 10 to about 50 nucleotides in length.

13. The mediator displacement probe of claim 10, wherein the target sequence specific portion comprises a region of complementarity to a target gene derived from a pathogen.

14. The mediator displacement probe of claim 10, wherein the label is selected from FAM, FITC, digoxigenin (DIG), and biotin.

15. The mediator displacement probe of claim 10, wherein the extension blocking agent is selected from biotin, a dehydroxylated 3′ nucleotide, 3′ dideoxycytosine (ddC), 3′ inverted deoxythymidine (dT), 3′ propyl (C3) spacer, 3′ amino modification, and a 3′ phosphoryl modification.

16. A mediator displacement probe set, the probe set comprising:
(i) a mediator displacement probe according to claim 10; and
(ii) a Universal Reporter hairpin polynucleotide having a 5′ end and a 3′ end, comprising a label attached to the 5′ end, and single stranded polynucleotide sequence that is complementary to the mediator (Med) polynucleotide sequence of the mediator displacement probe of (i) positioned at the 3′ end.

17. The mediator displacement probe set of claim 16, wherein the single stranded polynucleotide sequence complementary to the Med polynucleotide sequence is identical to the MedC portion of the mediator displacement probe.

18. A method for producing dual-labeled lateral flow assay (LFA) detection products, the method comprising
(a) performing an isothermal amplification reaction to amplify a target nucleic acid in the presence of a mediator displacement probe set, wherein the mediator displacement probe set comprises:
(i) a mediator displacement probe according to claim 10; and
(ii) a Universal Reporter hairpin polynucleotide having a 5′ end and a 3′ end, comprising a label attached to the 5′ end, and single stranded polynucleotide sequence that is complementary to the mediator (Med) polynucleotide sequence of the mediator displacement probe of (i) positioned at the 3′ end; and
(b) producing dual-labeled LFA detection products by integrating the mediator displacement probe into an amplicon of the isothermal amplification reaction, thereby releasing the Med portion of the mediator displacement probe, which subsequently anneals to the Universal Reporter hairpin polynucleotide to produce a dual-labeled LFA detection product.

19. The method of claim 18, wherein the isothermal amplification reaction is LAMP.

20. A method for indirectly detecting isothermal amplification of a target nucleic acid, the method comprising:
producing one or more dual-labeled LFA detection products according to the method of claim 18;
contacting a lateral flow assay (LFA) device with the one or more dual-labeled LFA amplification products to produce one or more detectable signals; and
identifying the presence of the target nucleic acid in the isothermal amplification based upon detecting the presence of the detectable signal produced by the one or more dual-labeled LFA detection products.
